(19) United States
(12) Patent Application Publication
(10) Pub. No.: US 2024/0306962 A1
(43) Pub. Date: Sep. 19, 2024

(54) Dermal Patch for Collecting a Physiological Sample

(71) Applicant: Satio, Inc., Boston, MA (US)

(72) Inventors: Namal NAWANA, Weston, MA (US); Mike MONIZ, Boston, MA (US); Ziad Tarilk AL-SHAMSIE, San Diego, CA (US)

(73) Assignee: Satio, Inc., Boston, MA (US)

(21) Appl. No.: 18/674,275

(22) Filed: May 24, 2024

Related U.S. Application Data
(63) Continuation-in-part of application No. 18/597,513, filed on Mar. 6, 2024.
Application No. 18/597,513 is a continuation-in-part of application No. 18/090,063, filed on Dec. 28, 2022.
Application No. 18/090,063 is a continuation-in-part of application No. 17/994,454, filed on Nov. 28, 2022, now Pat. No. 11,877,848.
Application No. 17/994,454 is a continuation of application No. 17/521,466, filed on Nov. 8, 2021, now Pat. No. 11,510,602.
Application No. 18/090,063 is a continuation-in-part of application No. 17/991,284, filed on Nov. 21, 2022, now Pat. No. 12,048,543.
Application No. 17/991,284 is a continuation-in-part of application No. 17/971,142, filed on Oct. 21, 2022, now Pat. No. 12,053,284.
Application No. 17/971,142 is a continuation-in-part of application No. 17/521,466, filed on Nov. 8, 2021, now Pat. No. 11,510,602.
Application No. 18/090,063 is a continuation-in-part of application No. 17/903,802, filed on Sep. 6, 2022.
Application No. 17/903,802 is a continuation of application No. 17/412,213, filed on Aug. 25, 2021, now Pat. No. 11,452,474.
Application No. 18/090,063 is a continuation-in-part of application No. 17/500,873, filed on Oct. 13, 2021, now Pat. No. 11,964,121.
Application No. 18/597,513 is a continuation-in-part of application No. 18/090,026, filed on Dec. 28, 2022, now Pat. No. 12,023,156.
Application No. 18/090,026 is a continuation-in-part of application No. 17/994,454, filed on Nov. 28, 2022, now Pat. No. 11,877,848.
Application No. 17/994,454 is a continuation-in-part of application No. 17/991,284, filed on Nov. 21, 2022, now Pat. No. 12,048,543.
Application No. 17/991,284 is a continuation-in-part of application No. 17/903,802, filed on Sep. 6, 2022.
Application No. 17/903,802 is a continuation-in-part of application No. 17/500,873, filed on Oct. 13, 2021, now Pat. No. 11,964,121.
Application No. 18/597,513 is a continuation-in-part of application No. 17/747,544, filed on May 18, 2022.
Application No. 18/597,513 is a continuation-in-part of application No. 17/719,881, filed on Apr. 13, 2022.
Application No. 18/597,513 is a continuation-in-part of application No. 17/412,205, filed on Aug. 25, 2021, now Pat. No. 12,029,562.
(63) Continuation-in-part of application No. 18/596,098, filed on Mar. 5, 2024.
Application No. 18/596,098 is a continuation-in-part of application No. 18/090,063, filed on Dec. 28, 2022.
Application No. 18/090,063 is a continuation-in-part of application No. 18/090,026, filed on Dec. 28, 2022, now Pat. No. 12,023,156.
Application No. 18/090,026 is a continuation-in-part of application No. 17/747,544, filed on May 18, 2022.
Application No. 17/747,544 is a continuation-in-part of application No. 17/719,881, filed on Apr. 13, 2022.
Application No. 17/719,881 is a continuation-in-part of application No. 17/412,205, filed on Aug. 25, 2021, now Pat. No. 12,029,562.
(60) Provisional application No. 63/190,700, filed on May 19, 2021.
(60) Provisional application No. 63/174,956, filed on Apr. 14, 2021.
(60) Provisional application No. 63/190,700, filed on May 19, 2021.
(60) Provisional application No. 63/174,956, filed on Apr. 14, 2021.
(60) Provisional application No. 63/190,700, filed on May 19, 2021.
(60) Provisional application No. 63/174,956, filed on Apr. 14, 2021.

(51) Int. Cl. A61B 5/15; A61F 13/84

(52) U.S. Cl. CPC A61B 5/150099; A61B 5/150206; A61B 5/150366; A61B 5/150755; A61F 13/84; A61F 2013/8473

(57) ABSTRACT

A system for collecting a physiological sample from a subject includes a lancet configured to draw a physiological sample and a cartridge configured to be affixed to the subject's skin. The cartridge includes a physiological sample well, a sloped physiological sample channel in open communication with the physiological sample well and a sample collection pad in in open communication with the sloped physiological channel and configured to absorb the drawn physiological sample. The physiological sample well is configured to retain the drawn physiological sample and the sloped physiological channel is configured to carry the drawn physiological sample from the physiological sample to the sample collection pad.

### RELATED APPLICATIONS

The present application claims priority to U.S. application Ser. No. 17/719,881 filed on Apr. 13, 2022 (4378-0010US01) which claims priority to U.S. Provisional Application No. 63/174,956 filed on Apr. 14, 2021 (4378-0010PV01), U.S. application Ser. No. 17/412,205 filed on Aug. 25, 2021 (4378-0013US01) which claims priority to U.S. Provisional Application No. 63/190,700 filed on May 19, 2021 (4378-0013PV01) and U.S. Provisional Application No. 63/174,956 filed on Apr. 14, 2021 (4378-0010PV01), U.S. application Ser. No. 17/903,802 filed on Sep. 6, 2022 (4378-0014US02) which claims priority to U.S. Provisional Application No. 63/190,700 filed on May 19, 2021 (4378-0013PV01), U.S. Provisional Application No. 63/174,956 filed on Apr. 14, 2021 (4378-0010PV01) and U.S. Provisional application Ser. No. 17/412,213 filed on Aug. 25, 2021 (4378-0014US01), U.S. application Ser. No. 18/090,026 filed on Dec. 28, 2022 (4378-0016US03) which claims priority to U.S. application Ser. No. 17/500,873 filed on Oct. 13, 2021 (4378-0015US01), U.S. application Ser. No. 17/903,802 filed on Sep. 6, 2022 (4378-0014US02), U.S. application Ser. No. 17/971,142 filed on Oct. 21, 2022 (4378-0018US01), U.S. application Ser. No. 17/991,284 filed on Nov. 21, 2022 (4378-0019US01) and U.S. application Ser. No. 17/994,454 filed on Nov. 28, 2022 (4378-0016US02), U.S. application Ser. No. 17/971,142 filed on Oct. 21, 2022 (4378-0018US01) which claims priority to U.S. application Ser. No. 17/521,466 filed on Nov. 8, 2021 (4378-0016US01), U.S. application Ser. No. 18/090,063 filed on Dec. 28, 2022 (4378-0020US01) which claims priority to U.S. application Ser. No. 17/500,873 filed on Oct. 13, 2021 (4378-0015US01), U.S. application Ser. No. 17/903,802 filed on Sep. 6, 2022 (4378-0014US02), U.S. application Ser. No. 17/971,142 filed on Oct. 21, 2022 (4378-0018US01), U.S. application Ser. No. 17/991,284 filed on Nov. 21, 2022 (4378-0019US01) and U.S. application Ser. No. 17/994,454 filed on Nov. 28, 2022 (4378-0016US02), U.S. application Ser. No. 18/597,513 filed on Mar. 6, 2024 (4378-0022US01) which claims priority to U.S. Provisional Application No. 63/174,956 filed on Apr. 14, 2021 (4378-0010PV01), U.S. Provisional No. 63/190,700 filed on May 19, 2021 (4378-0013PV01), U.S. application Ser. No. 17/412,205 filed on Aug. 25, 2021 (4378-0013US01), U.S. application Ser. No. 17/719,881 filed on Apr. 13, 2022 (4378-0010US01), U.S. application Ser. No. 17/747,544 filed on May 18, 2022 (4378-0013US02), U.S. application Ser. No. 17/903,802 filed on Sep. 6, 2022 (4378-0014US02), U.S. application Ser. No. 17/971,142 filed on Oct. 21, 2022 (4378-0018US01), Ser. No. 17/991,284 filed on Nov. 21, 2022 (4378-0019US01), U.S. application Ser. No. 18/090,026 filed on Dec. 28, 2022 (4378-0016US03) and U.S. application Ser. No. 18/090,063 filed on Dec. 28, 2022 (4378-0020US01), U.S. application Ser. No. 18/596,098 filed on Mar. 5, 2024 (4378-0024US01) which claims priority to U.S. Application No. 63/174,956 filed on Apr. 14, 2021 (4378-0010PV01), U.S. Provisional Application No. 63/190,700 filed May 19, 2021 (4378-0013PV01), U.S. application Ser. No. 17/412,205 filed on Aug. 25, 2021 (4378-0013US01), U.S. application Ser. No. 17/412,213 filed on Aug. 25, 2021 (4378-0014US01), U.S. application Ser. No. 17/500,873 filed on Oct. 13, 2021 (4378-0015US01), U.S. application Ser. No. 17/521,466 filed on Nov. 8, 2021 (4378-0016US01), U.S. application Ser. No. 17/719,881 filed on Apr. 13, 2022 (4378-0010US01), U.S. application Ser. No. 17/747,544 filed May 18, 2022 (4378-0013US02), U.S. application Ser. No. 17/903,802 filed on Sep. 6, 2022 (4378-0014US02), U.S. application Ser. No. 17/971,142 filed on Oct. 21, 2022 (4378-0018US01), U.S. application Ser. No. 17/991,284 filed on Nov. 21, 2022 (4378-0019US01), U.S. application Ser. No. 17/994,454 filed Nov. 28, 2022 (4378-0016US02), U.S. application Ser. No. 18/090,026 filed on Dec. 28, 2022 (4378-0016US03) and U.S. application Ser. No. 18/090,063 filed on Dec. 28, 2022 (4378-0020US01).

The present application is also related to utility application entitled Dermal Patch for Collecting a Physiological Sample which is filed concurrently herewith and is hereby incorporated by reference in its entirety.

### TECHNICAL FIELD

The present teachings are generally directed to a lancet for use with a cartridge of a dermal patch system (also referred to as “dermal patches” herein).

### BACKGROUND

Several diagnostic tests may be completed by analyzing a physiological sample from a subject. In order to obtain the physiological sample, a subject may have to travel to a diagnostic lab, wherein a medical professional obtains the sample with a standard syringe. This process may be time consuming and burdensome to the subject. At home solutions, may allow a user to collect the sample with a needle that, after use, may not be properly disposed of which may result in accidental injury.

### SUMMARY

Aspects of the present disclosure address the above-referenced problems and/or others and satisfy an unmet need for a safe lancet that can draw a physiological sample from a subject.

In one aspect, a system for collecting a physiological sample from a subject includes a lancet configured to draw a physiological sample, and a cartridge configured to be affixed to the subject's skin. The cartridge includes a physiological sample well, a sloped physiological sample channel in open communication with the physiological sample well, and a sample collection pad in open communication with the sloped physiological channel and configured to absorb the drawn physiological sample. The physiological sample well is configured to retain the drawn physiological sample and the sloped physiological channel is configured to carry the drawn physiological sample from the physiological sample well to the sample collection pad. In some embodiments the system can include a desiccant disposed within the cartridge, but a desiccant is not necessary. In some embodiments, the system further includes a hydrophobic foam disposed within the cartridge and configured to prevent the drawn physiological sample from escaping the sample collection pad. In some embodiments, the sample collection pad can comprise sections with tabs therebetween. In some embodiments, the cartridge can comprise a bottom layer, an adhesive layer attached to the bottom layer, a top layer, a sample well opening formed in the adhesive layer, a needle aperture formed in the top layer, wherein the needle aperture is smaller than the sample well opening. The desiccant can be formed in a space between the top layer and the bottom layer of the cartridge. The adhesive layer can be formed of a pressure sensitive adhesive, and can include at least one cutout vent hole configured to allow the physiological sample on the sample collection pad to dry faster. The top layer can include a cutout configured to hold an integrated overflow filter.

In some embodiments, the sample collection pad is a first sample collection pad and the cartridge includes a second collection pad. The physiological sample channel is configured to carry the drawn physiological sample to the first and second collection pad. In some embodiments, the lancet includes one or more needles configured to move between an undeployed position and a deployed position, wherein the one or more needles are disposed within the lancet when in the undeployed position and extends out of the lancet when in the deployed position to draw the physiological sample. In some embodiments, the cartridge includes a lancet receiving element. The lancet is configured to move the one or more needles from the undeployed position to the deployed position when the lancet engages with the lancet receiving element, or when a configuration of the lancet engages with a matching configuration of the cartridge. In some embodiments, the lancet is configured to automatically retract one or more needles into a housing of the lancet. In some embodiments, the system includes a vacuum pin disposed within the cartridge and configured to move between a deployed position and an undeployed position. The vacuum pin is configured to create a vacuum within the cartridge when moved from the undeployed position to the deployed position and the vacuum draws the drawn physiological sample from the physiological sample well to the sample collection pad. In some embodiments, the cartridge includes a first layer of material, and a second layer of material. The first and second layer of material define the physiological sample channel.

In another aspect, a system for collecting a physiological sample from a subject includes a lancet configured to draw a physiological sample and a cartridge configured to receive and store the physiological sample. The cartridge includes a cover with a first portion and a second portion, wherein the first portion is removable, a first quick response code disposed on the first portion of the cover, and a second quick response code disposed on the second portion. The first and second quick response codes are associated with the cartridge. In some embodiments, the cartridge further includes a sample collection pad. The cover seals the sample collection pad within the cartridge. In some embodiments, the cover includes a transparent portion that allows a user to view the sample collection pad. In some embodiments, the cover includes a third portion that covers the sample collection pad and includes the transparent portion, wherein the third portion is removable.

In some embodiments, the lancet includes one or more needles that are moveable between an undeployed position and a deployed position. The one or more needles are configured to draw the physiological sample when in the deployed position. The cartridge includes a lancet receiving element configured to cause the lancet to move the one or more needles to the deployed position. The first portion covers the lancet receiving element. In some embodiments, the first portion includes a pull tab which allows a user to remove the first portion. In some embodiments, the cartridge includes a moveable vacuum pin configured to create a vacuum within the cartridge when moved. The vacuum draws the physiological sample to the collection pad.

In another aspect, a method for drawing a physiological sample includes affixing a cartridge of a dermal patch system to the skin of a subject, engaging a lancet with the cartridge to draw a physiological sample, removing the cartridge from the skin of the subject, and determining if chain of custody of the cartridge has been preserved by scanning a quick response code of the cartridge. In some embodiments, the lancet includes one or more needles that are configured to move from an undeployed position to a deployed position upon engagement with the cartridge. In some embodiments, the lancet is configured to automatically retract the one or more needles. In some embodiments, the method further includes pulling a vacuum pin of the cartridge to draw the physiological sample to a collection pad of the cartridge.

In another embodiment, the one or more needles can be coated with silicone. In another embodiment, the cartridge can include alignment ridges to align the bottom layer of the cartridge with the top layer of the cartridge. In another embodiment, the collection pad can be made from one piece having tabs formed at a middle portion thereof, thereby forming a first section and a second section that are configured to be separated from each other when the first and second section are pulled apart. The first section and the second section each can be approximately 8 mm by 8 mm, or approximately 16 mm by 15 mm.

In another embodiment, the sample collection pad can be configured to separate a plasma sample from the physiological sample. In another embodiment, the sample collection pad can comprise a blood separation section and a conjugate release section. The blood separation section can comprise a separator selected from a group consisting of an LF1 separator, an MF1 separator, a VF2 vertical separator, a Fusion 5 separator, and GF/DVA bound glass separator. The conjugate release section can comprise a conjugate release element selected from a group consisting of a Std 14, Std 17 and Fusion 5. In another embodiment, the sample collection pad can comprise a plasma separation stack up. The plasma separation stack up can comprise a membrane configured to remove white blood cells, a membrane configured to remove red blood cells and a layer configured to provide a wicking source and plasma storage. In another embodiment, the plasma separation stack up comprises top clear laminate layer configured to allow viewing of the plasma sample, a polyester membrane located below the top clear laminate layer and configured to remove white blood cells, a first double-sided adhesive located below the polyester membrane and configured to adhere to the polyester membrane, an asymmetric polysulfone layer located below the first double-sided adhesive and configured to remove red blood cells (RBC's), a second double-sided adhesive located below the asymmetric polysulfone layer and configured to adhere to the asymmetric polysulfone layer, a wax-patterned cellulose layer located below the second double-sided adhesive and configured to provide a wicking source and plasma storage, a third double-sided adhesive located below the wax-patterned cellulose layer and configured to adhere to the wax-patterned cellulose layer, and a clear laminate bottom layer located below the third double-sided adhesive. Another embodiment comprises an overflow area in the vicinity of the pad configured to limit the saturation of blood on the pad in order to keep the collected blood volume within a targeted range of blood volume.

### BRIEF DESCRIPTION OF THE DRAWINGS

Aspects of the present disclosure may take form in various components and arrangements of components, and in various steps and arrangements of steps. The drawings are only for illustration purpose of preferred embodiments of the present disclosure and are not to be considered as limiting.

Features of embodiments of the present disclosure will be more readily understood from the following detailed description take in conjunction with the accompanying drawings in which:

FIG. 1 depicts a dermal patch system in accordance with an exemplary embodiment of the present disclosure;

FIG. 2 depicts a lancet with three needles in accordance with an exemplary embodiment of the present disclosure;

FIG. 3 is a cross sectional view of the lancet, wherein the lancet is in an undeployed position in accordance with an exemplary embodiment of the present disclosure;

FIG. 4 is a cross sectional view of a housing of the lancet in accordance with an exemplary embodiment of the present disclosure;

FIG. 5 shows an inner cap of the housing in accordance with an exemplary embodiment of the present disclosure;

FIG. 6 depicts a side of an inner sleeve of the lancet in accordance with an exemplary embodiment of the present disclosure;

FIG. 7 depicts another side of the inner sleeve of the lancet in accordance with an exemplary embodiment of the present disclosure;

FIG. 8 is a cross sectional view of the inner sleeve of the lancet in accordance with an exemplary embodiment of the present disclosure;

FIG. 9 depicts a side of a needle platform of the lancet in accordance with an exemplary embodiment of the present disclosure;

FIG. 10 depicts another side of the needle platform in accordance with an exemplary embodiment of the present disclosure;

FIG. 11 is a cross sectional view of a spring sleeve of the lancet in accordance with an exemplary embodiment of the present disclosure;

FIG. 12 depicts a cartridge of the dermal patch system in accordance with an exemplary embodiment of the present disclosure;

FIG. 13 is an exploded view of the cartridge in accordance with an exemplary embodiment of the present disclosure;

FIG. 14 depicts an adhesive layer of the cartridge in accordance with an exemplary embodiment of the present disclosure;

FIG. 15 depicts a bottom layer of the cartridge in accordance with an exemplary embodiment of the present disclosure;

FIG. 16 depicts a collection pad support of the bottom layer cartridge in accordance with an exemplary embodiment of the present disclosure;

FIG. 17 depicts a desiccant of the cartridge in accordance with an exemplary embodiment of the present disclosure;

FIG. 18 depicts a top layer of the cartridge in accordance with an exemplary embodiment of the present disclosure;

FIGS. 19A-19C show another depiction of the top layer of the cartridge in accordance with an exemplary embodiment of the present disclosure;

FIG. 20 is a bottom view of the top layer of the cartridge in accordance with an exemplary embodiment of the present disclosure;

FIG. 21 depicts a lancet receiving element of the top layer in accordance with an exemplary embodiment of the present disclosure;

FIG. 22 depicts the cartridge of the dermal patch system without a cover and collection pads in accordance with an exemplary embodiment of the present disclosure;

FIG. 23 is a cross sectional view of the cartridge in accordance with an exemplary embodiment of the present disclosure;

FIG. 24 depicts a vacuum pin of the cartridge in accordance with an exemplary embodiment of the present disclosure;

FIG. 25 depicts a cover of the dermal patch system in accordance with an exemplary embodiment of the present disclosure;

FIG. 26 depicts the cartridge of the dermal patch system with a portion of the cover removed in accordance with an exemplary embodiment of the present disclosure;

FIG. 27 is a cross sectional view of the lancet engaging with the cartridge in accordance with an exemplary embodiment of the present disclosure;

FIG. 28 is a cross sectional of the lancet transitioning to a deployed position in accordance with an exemplary embodiment of the present disclosure;

FIG. 29 is a cross sectional of the lancet transitioning to a deployed position in accordance with an exemplary embodiment of the present disclosure;

FIG. 30 is a cross sectional view of the lancet engaging with the cartridge, wherein the lancet is in a deployed position in accordance with an exemplary embodiment of the present disclosure;

FIG. 31 is a cross sectional view of the lancet in a deployed position in accordance with an exemplary embodiment of the present disclosure;

FIG. 32 is a cross sectional view of the lancet engaging with the cartridge, wherein the lancet is in a retracted position in accordance with an exemplary embodiment of the present disclosure;

FIG. 33 is a cross sectional view of the lancet, wherein the lancet is in a retracted position in accordance with an exemplary embodiment of the present disclosure;

FIG. 34 is a cross sectional view of the lancet engaging with the cartridge, wherein the lancet is in a retracted position in accordance with an exemplary embodiment of the present disclosure;

FIG. 345 depicts the cartridge of the dermal patch system including two quick response (QR) codes in accordance with an exemplary embodiment of the present disclosure;

FIG. 36 depicts a computer system scanning the quick response codes of the cartridge in accordance with an exemplary embodiment of the present disclosure;

FIG. 37 depicts the computer system connected to a quick response code database in accordance with an exemplary embodiment of the present disclosure;

FIG. 38 depicts a first computer system scanning a first quick response code of the cartridge and a second computer system scanning a second quick response code of the cartridge in accordance with an exemplary embodiment of the present disclosure;

FIG. 39 is a flow chart of a method for obtaining a physiological sample in accordance with an exemplary embodiment of the present disclosure;

FIG. 40 schematically depicts a computer system in accordance with an exemplary embodiment of the present disclosure; and

FIG. 41 schematically depicts a cloud computing environment in accordance with an exemplary embodiment;

FIG. 42 shows example geometry and needle dimensions of an illustrative embodiment;

FIG. 43 depicts a bottom layer of the cartridge in accordance with another embodiment of the present disclosure;

FIG. 44 depicts a top layer of the cartridge in accordance with another embodiment of the present disclosure;

FIG. 45 depicts a collection pad in accordance with another embodiment of the present disclosure.

FIG. 46 shows a collection pad that can collect a larger physiological sample.

FIG. 47 shows an example plasma filter.

FIG. 48 shows a sample collection pad that can be used to collect plasma.

### DETAILED DESCRIPTION

The present disclosure generally relates to a dermal patch system (which may also be referred to as a “dermal patch”) that may be utilized to collect and optionally store a physiological sample.

In various embodiments, a dermal patch system may be used to collect a physiological sample and the collected sample may then be stored on a sample collection pad of the dermal patch system. Dermal patch systems disclosed herein may allow for the collection and analysis of a physiological sample in a variety of environments (e.g., at home, in the field, in a medical facility, etc.).

The term “about,” as used herein, denotes a deviation of at most 10% relative to a numerical value. For example, about 100 m means in the range of 90 m-110 m.

The term “substantially,” as used herein, refers to a deviation, if any, of at most 10% from a complete state and/or condition.

The term “majority,” as used herein, refers to more than 50% of an object or substance.

The term “subject” as used herein refers to a human subject or an animal subject (i.e., chicken, pig, cattle, dog, cat, etc.).

The term “transparent,” as used herein, indicates that light can substantially pass through an object (e.g., a window) to allow visualization of a material disposed behind the object. For example, in some embodiments, a transparent object allows the passage of at least 70%, or at least 80%, or at least 90% of visible light therethrough.

The term “needle” as used herein, refers to a component with a pointed tip that is configured to pierce an outer surface of an element (e.g., skin of a subject) to provide a passageway through which a physiological fluid can be extracted.

The term “vacuum,” as used herein, refers to a pressure less than atmospheric pressure and more particularly to a pressure that can facilitate the movement of a fluid (e.g., a physiological sample) within a dermal patch system according to various embodiments.

The present disclosure generally relates to a device, which is herein also referred to as a dermal patch or a dermal patch system, for collecting a physiological sample (e.g., bodily fluids such as blood, interstitial fluids, etc.) from a subject. In some embodiments discussed below, such a dermal patch system can include a cartridge that can be affixed to a subject's skin (e.g., via an adhesive layer) and a separate lancet that can be engaged with the cartridge to puncture the skin, thereby providing a passageway for extracting the physiological sample. As discussed in more detail below, the lancet can include a housing in which at least one needle that is configured for puncturing the skin is disposed. The lancet can be transitioned between at least three states. In a first state (which may be referred to as an undeployed or a locked state) the lancet retains the needles within the housing when the lancet is not engaged with the cartridge. In a second state (which may be referred to as a deployed, an unlocked state, or a released state) the lancet allows the needles to puncture the skin in response to engagement of the lancet with the cartridge. In a third state (which may be referred to as a retracted state) the lancet automatically moves the needles from the deployed position back into the lancet housing. In other words, the engagement of the lancet with the cartridge automatically transitions the lancet from the locked state to the unlocked state, and to the retracted state. In some embodiments, the lancet includes an inner sleeve with a plurality of deformable tabs and a spring sleeve. The spring sleeve retains a lower spring in a compressed state and the deformable tabs contact the spring sleeve. This retains the spring sleeve in a first position. In the first position, the spring sleeve compresses the lower spring and prevents the lower spring from expanding.

When the lancet is engaged with the cartridge, the inner sleeve moves from a first position to a second position. In the first position, the inner sleeve retains a needle platform that supports the needles in an undeployed position. The lancet also includes an upper spring that is coupled to the needle platform. When the needle platform is in the undeployed position, the needle platform compresses the upper spring and prevents the upper spring from expanding. In the second position, the inner sleeve moves vertically upward which releases the needle platform thereby allowing the needle platform to move to a deployed position. When released, the upper spring is allowed to expand which provides a force to the needle platform and the needles extend beyond the housing of the lancet. Furthermore, this force causes the needles (which are coupled to the needle platform) to travel with sufficient velocity to puncture the skin of a subject. As the needle platform travels to the deployed position, the needle platform contacts the deformable tabs and causes the tabs to break or deflect. When broken (or deflected) the tabs no longer contact the spring sleeve which allows the lower spring to expand and move the spring sleeve vertically upward from a first position to a second position which moves the needle spring vertically upward such that the needles are retracted into the housing of the lancet.

In this manner, the lancet remains safe before it is engaged with the cartridge as the lancet is not capable of deploying the needles when the lancet is not engaged with the cartridge. Furthermore, in this manner, the lancet remains safe after drawing a physiological sample as the needles automatically retract back into the lancet's housing after being deployed.

After the physiological sample is drawn, a user of the dermal patch system pulls a vacuum pin of the dermal patch system which creates a vacuum within the cartridge. This vacuum draws the physiological sample to a collection pad of the cartridge. The collection pad retains and stores the physiological sample.

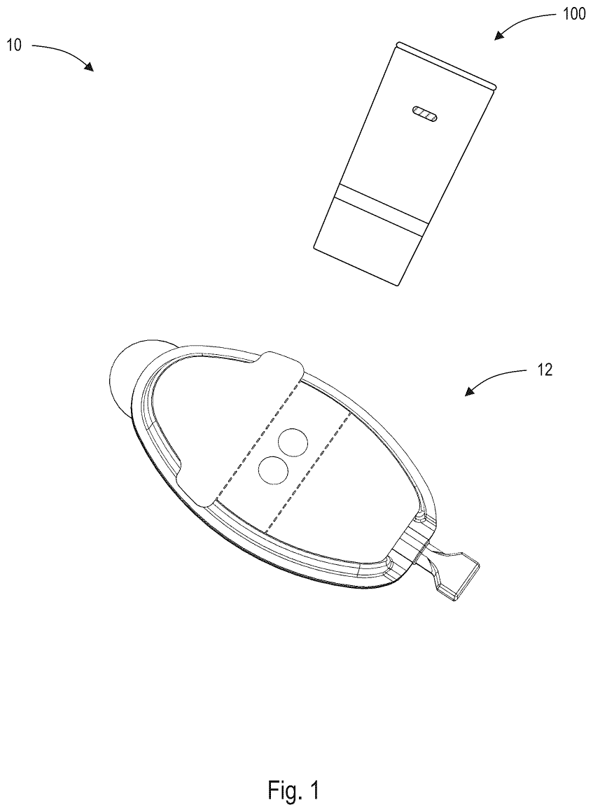

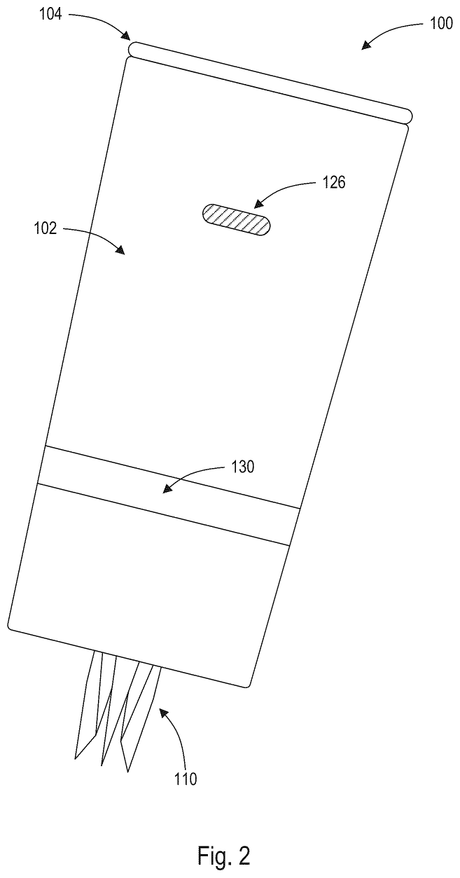

Referring now to FIGS. 1 and 2, a dermal patch system 10 is shown in accordance with an exemplary embodiment. In this embodiment, the dermal patch system 10 includes a cartridge 12 that can be affixed to a subject's skin and a lancet 100.

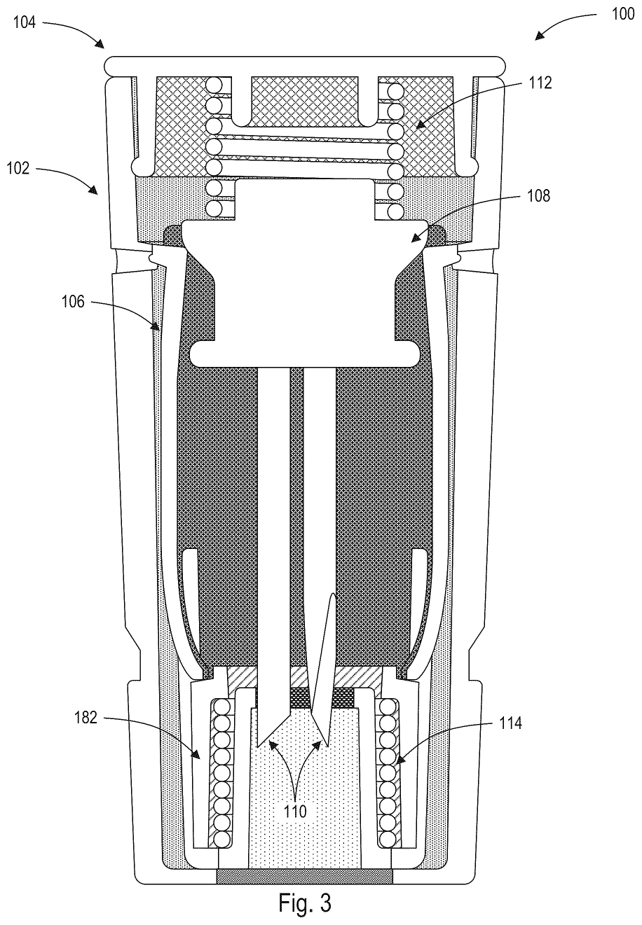

With reference to FIG. 3 a cross sectional view of the lancet 100 is shown in accordance with an exemplary embodiment. The lancet 100 includes a housing 102 and a cap 104 that is coupled to the housing 102. The housing 102 and the cap 104 define an inner volume of the lancet 100 in which various components of the lancet 100 are disposed. The lancet 100 further includes an inner sleeve 106 and a needle platform 108 that are disposed within the inner volume of the lancet 100. The needle platform 108 supports a plurality of needles 110, specifically three needles 110. While needle platform 108 is depicted as supporting three needles 110 (FIG. 2), in other embodiments, the needle platform 108 can support more or less needles 110 (e.g., 1, 2, 4, 5, needles etc.). The lancet 100 further includes an injection spring (also referred to as an upper spring) 112 and a retraction spring (also referred to as a lower spring) 114 that move the needle platform 108.

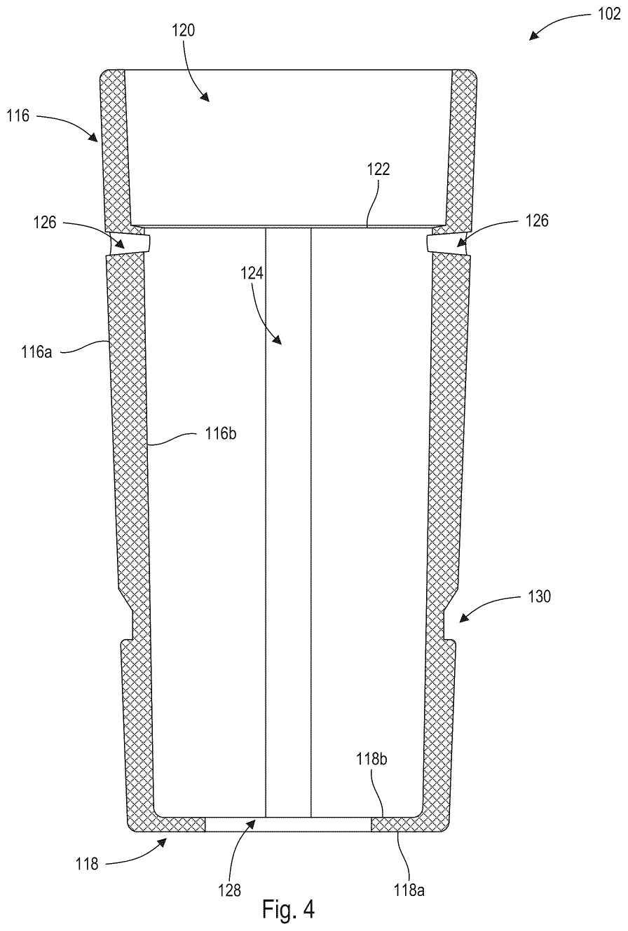

With particular reference to FIG. 4, the housing 102 includes a side wall 116 and a bottom wall 118. The side wall 116 includes an outer surface 116a and an opposed inner surface 116b. The bottom wall 118 includes an outer surface 118a and an opposed inner surface 118b. The side wall 116 extends substantially vertically from the bottom wall 118. The side wall 116 and the bottom wall 118 are generally cylindrical in shape and are concentric with one another relative to a longitudinal axis of the side wall 116. The inner surface 116b of the side wall 116 and the inner surface 118b of the bottom wall 118 define an inner volume 120. An upper portion of the inner surface 116b of the side wall 116 is wider than a lower portion of the inner surface 116b such that the inner surface 116b defines a ledge 122. The inner surface 116b also defines a first and second groove 124 that are opposed from one another (only one of which is shown in the cross section in FIG. 4). The grooves 124 extend between the ledge 122 and the inner surface 118b of the bottom wall 118.

The side wall 116 defines openings 126 that extend through the side wall 116. That is, the openings 126 extend between the outer surface 116a and the inner surface 116b of the side wall 116. The bottom wall 118 defines an aperture 128 that extends through the bottom wall 118. That is, the aperture 128 extends between the outer surface 118a and the inner surface 118b of the bottom wall 118. As will be discussed in further detail herein, when the lancet 100 is activated via engagement with the cartridge 12, the needles of the lancet 100 extend through the aperture 128 for puncturing a subject's skin to draw a physiological sample. The outer surface 118 of the side wall 116 further defines an outer groove 130. As will be discussed in further detail herein, a portion of the cartridge 12 engages with the groove 130 to couple the lancet 100 to the cartridge 12.

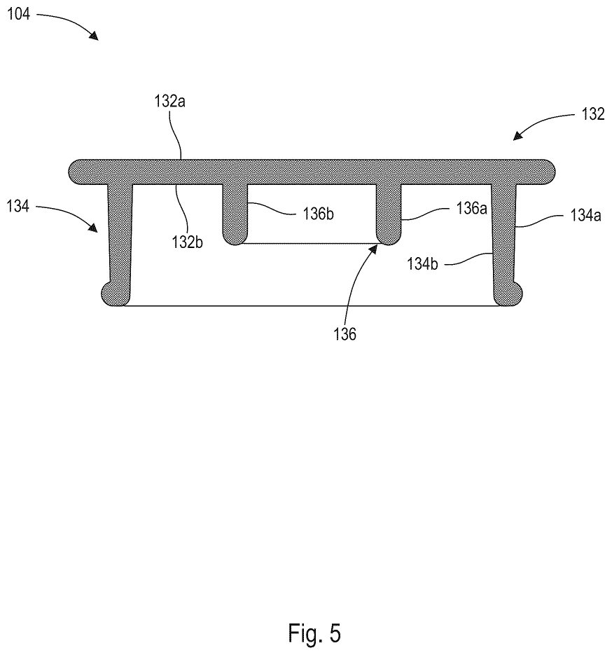

Referring now to FIG. 5, the inner cap 104 is shown in accordance with an exemplary embodiment. The cap 104 includes a top wall 132 with an outer surface 132a and an opposed inner surface 132b. The cap 104 also includes a side wall 134 with an outer surface 134a and an opposed inner surface 134b. The top wall 132 extends substantially horizontally from and perpendicular to the side wall 134. The side wall 134 extends substantially vertically from and perpendicular to the top wall 132. The top wall 132 and the side wall 134 are generally circular in shape and are generally concentric with one another. The cap 104 also includes an inner cylinder 136 with an outer surface 136a and an opposed inner surface 136b. Inner cylinder 136 extends substantially vertically from and perpendicular to the top wall 132. The inner cylinder 136 is generally concentric with the top wall 132 and the side wall 134. The inner cylinder 136 extends substantially vertically from and perpendicular to the top wall 132. The inner cylinder 136 is concentric with the top wall 132 and the side wall 134.

When the cap 104 is coupled to the housing 102 the side wall 134 extends into the inner volume 120 of the housing 102 and at least a portion of the side wall 134 contacts the inner surface 116b of the side wall 116 such that the cap 104 couples to the housing 102 via an interference fit.

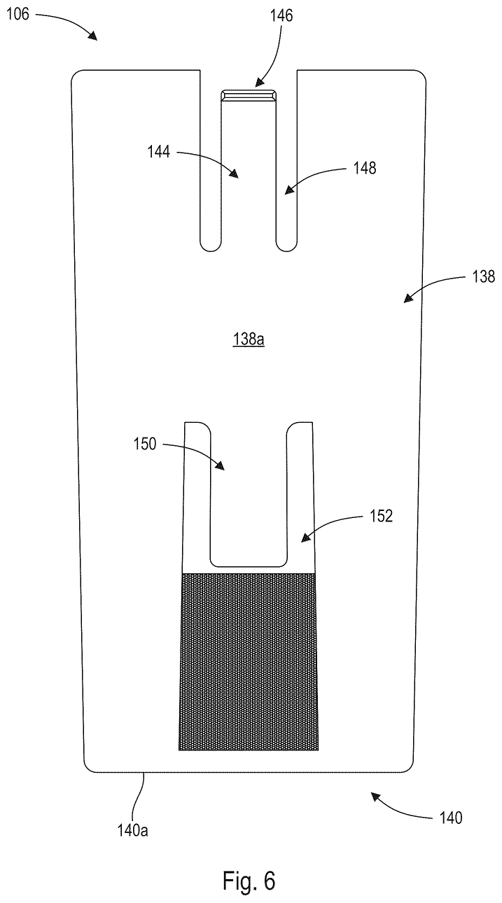

Referring now to FIGS. 6-8, the inner sleeve 106 is shown in accordance with an exemplary embodiment. The inner sleeve 106 includes a side wall 138 and a bottom wall 140. The side wall 138 includes an outer surface 138a and an opposed inner surface 138b. The bottom wall 140 includes an outer surface 140a and an opposed inner surface 140b. The side wall 138 extends substantially vertically from the bottom wall 140. The side wall 138 and the bottom wall 140 are generally circular in shape and are concentric with one another. The inner surface 138b of the side wall 138 and the inner surface 140b of the bottom wall 140 define an inner volume 142.

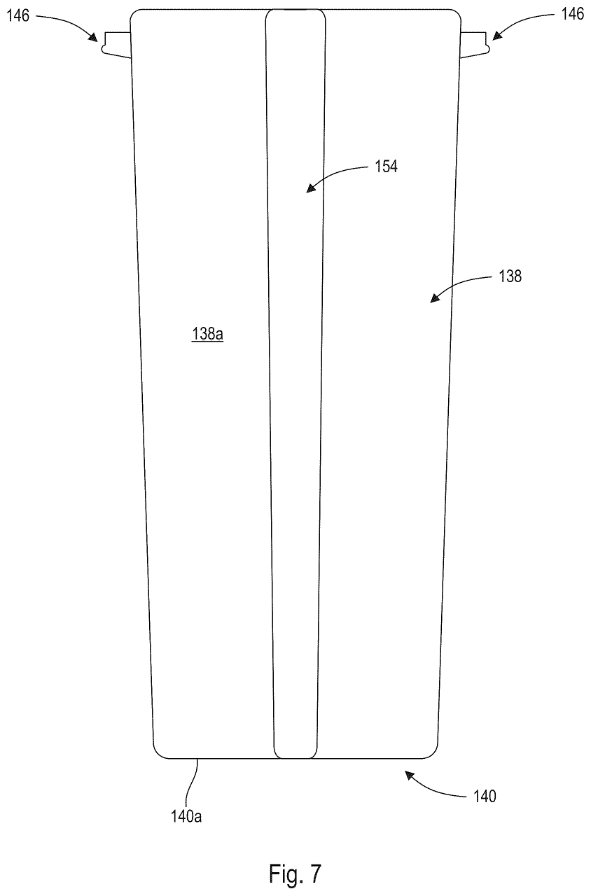

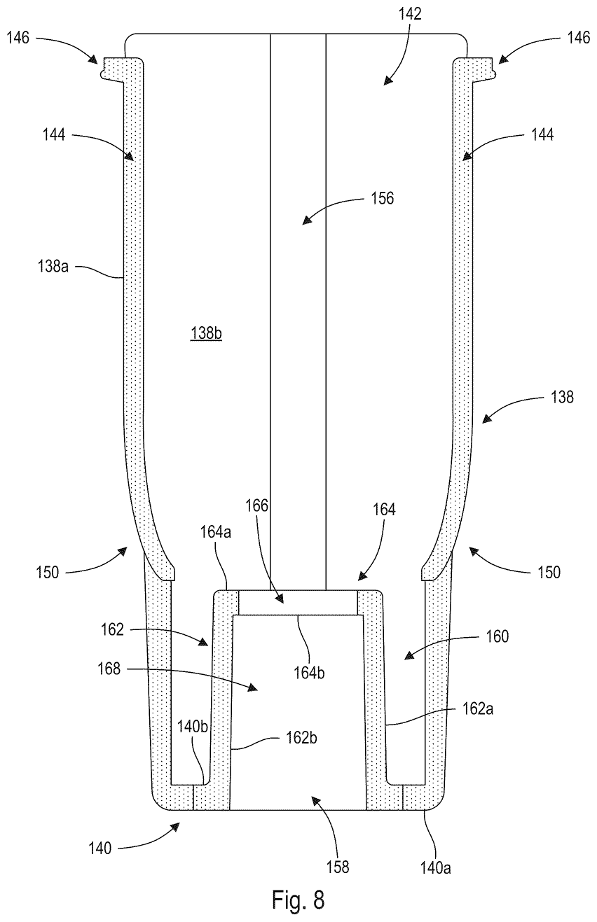

The side wall 138 defines a first and second deformable extension 144 that extend within a gap 148 of the side wall 138. The extensions 144 each include a hook 146. As will be discussed in further detail herein, the hooks 146 of the extensions 144 extend into the openings 126 of the housing 102 which retains the lancet 100 in the undeployed position. The side wall 138 further defines a first and second deformable tab 150 that extend within an opening 152 of the side wall 138. The side wall 138 also includes a first and second protrusion 154 (only one of which is shown in FIG. 7) that extend substantially horizontally from and perpendicular to the outer surface 138a of the side wall 138. When the inner sleeve 106 is disposed within the housing 102, the protrusions 154 extend within the grooves 124 of the housing 102. When the inner sleeve 106 is moving between positions (e.g., between an undeployed position to a deployed position), the grooves 124 and the protrusions 154 guide the vertical movement of the inner sleeve 106 while preventing a rotational or horizontal movement of the inner sleeve 106. The inner surface 138b of the side wall 138 further defines a first and second groove 156 that are opposed from one another (only one of which is shown in FIG. 8). The grooves 156 extend substantially vertically from and perpendicular to the inner surface 140b of the bottom wall 140.

The inner sleeve 106 includes an opening 158 that extends through the bottom wall 140. That is, the opening 158 extends between the outer surface 140a and the inner surface 140b of the bottom wall 140. The inner sleeve 106 also includes a post 160 that is cylindrical in shape and is generally concentric with the opening 158. The post 160 includes a side wall 162 and a top wall 164. The side wall 162 extends substantially vertically from and perpendicular to the inner surface 140b of the bottom wall 140. The side wall 162 includes an outer surface 162a and an opposed inner surface 162b. The top wall 164 extends substantially horizontally between the side wall 162 and includes a top surface 164a and an opposed bottom surface 164b. The top wall 164 includes an opening 166 that extends through the top wall 164. That is, the opening 166 extends between the top surface 164a and the bottom surface 164b of the top wall 164. The inner surface 162b of the side wall 162 and the bottom surface 164b of the top wall 164 define an inner volume 168 of the post 160. As will be discussed in further detail herein, the needles 110 extend through the opening 166 of the top wall 164, through the inner volume 168, and through the opening 158 of the bottom wall 140 when in the deployed position.

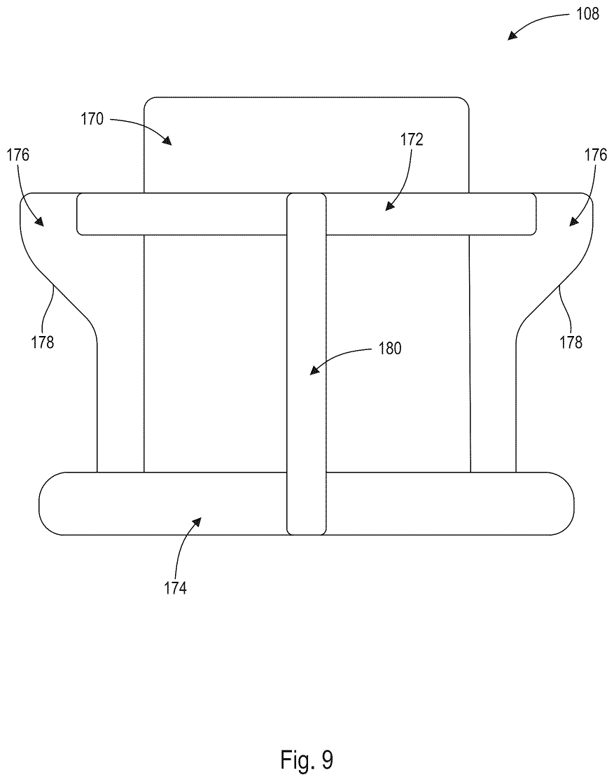

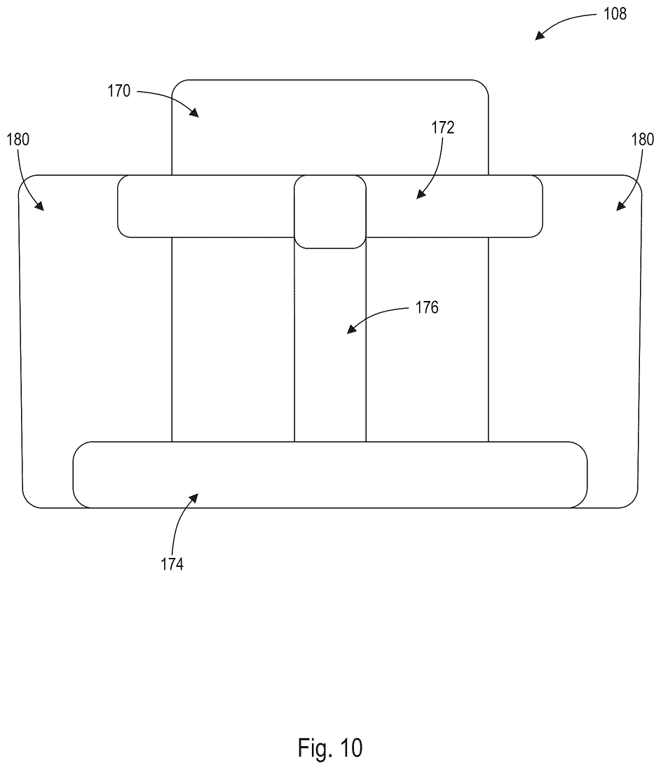

Referring now to FIGS. 9 and 10, the needle platform 108 is shown in accordance with an exemplary embodiment. The needle platform 108 includes a cylindrical body 170, an upper circular extension 172 and a lower circular extension 174. The upper circular extension 172 and the lower circular extension 174 extend substantially horizontally from and perpendicular to the cylindrical body 170. As will be discussed in further detail herein, the upper circular extension 172 serves as a platform for the injection spring 112. The needle platform 108 further includes a first and second curved extension 176. The curved extensions 176 extend substantially horizontally from and perpendicular to the cylindrical body 170 and extend substantially vertically between the upper circular extension 172 and the lower circular extension 174. Each curved extension 176 includes an angled surface 178. As will be discussed in further detail herein, when in the undeployed position, the angled surfaces 178 contact the hooks 146 of the inner sleeve 106 which prevents the needle platform 108 from transitioning to the deployed position. The needle platform 108 further includes a first and second a projection 180. The first and second projection 180 are generally rectangular in shape, extend substantially horizontally from and perpendicular to the cylindrical body 170, and extend substantially vertically between the upper circular extension 172 and the lower circular extension 174. When the needle platform 108 is disposed within the inner sleeve 106, the projections 180 extend into the grooves 156 which guides the vertical movement of the needle platform 108 while preventing a rotational or horizontal movement of the needle platform 108 needle platform 108.

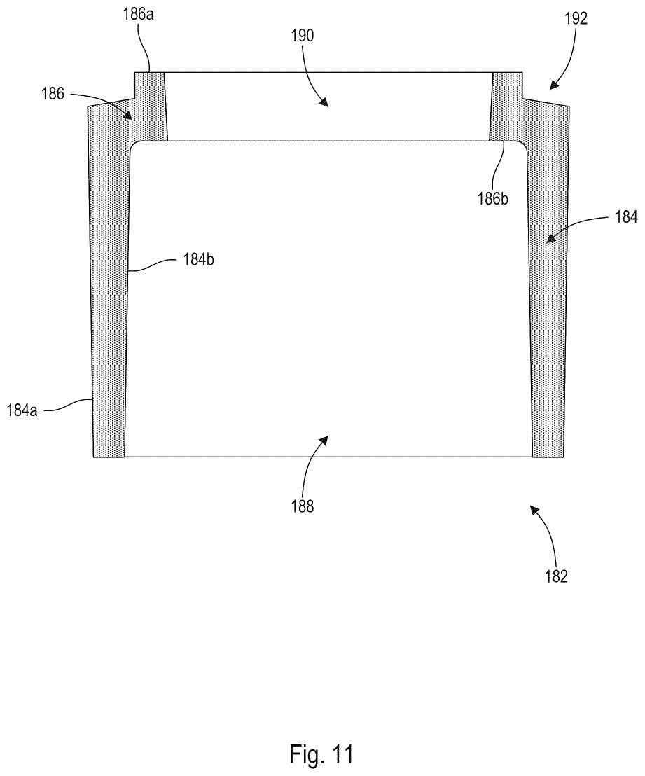

As depicted in FIG. 3, the lancet 100 further includes a spring sleeve 182. With particular reference to FIG. 11, the spring sleeve 182 is shown in accordance with an exemplary embodiment. The spring sleeve 182 is cylindrical in shape and includes a side wall 184 and a top wall 186. The side wall 184 includes an outer surface 184a and an opposed inner surface 184b and the top wall 186 includes a top surface 186a and an opposed bottom surface 186b. The inner surface 184b of the side wall 184 and the bottom surface 186b of the top wall 186 define an inner volume 188 of the spring sleeve 182. The side wall 184 and the top wall 186 are shaped and dimensioned such that the spring sleeve 182 retains the retraction spring 114 within the inner volume 188. The top wall 186 includes an opening 190 that extends therethrough. That is, the opening 190 extends between the top surface 186a and the bottom surface 186b of the top wall 186. The opening 190 of the top wall 186 is in communication with the inner volume 188. Since the inner volume 188 is open, the needles 110 may extend through spring sleeve 182 via the opening 190. The top surface 184a defines a ledge 192 that extends circumferentially around the spring sleeve 182. As will be discussed in further detail herein, when the lancet 100 is in the undeployed position, the tabs 150 contact the ledge 192.

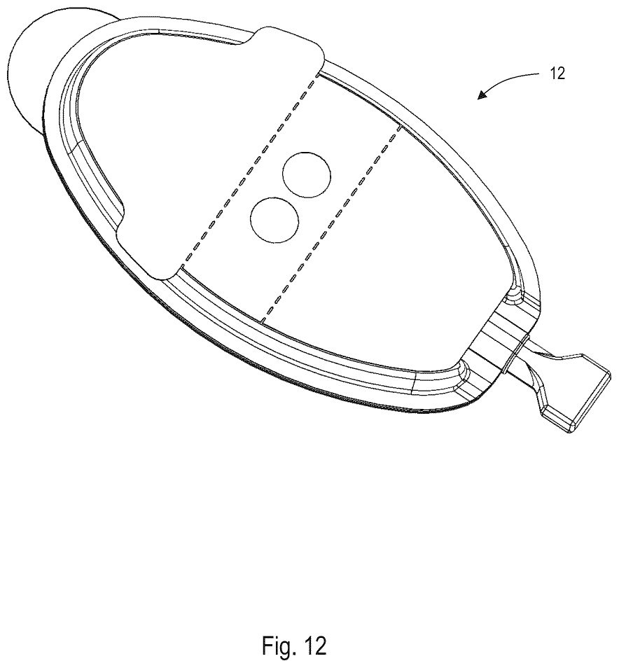

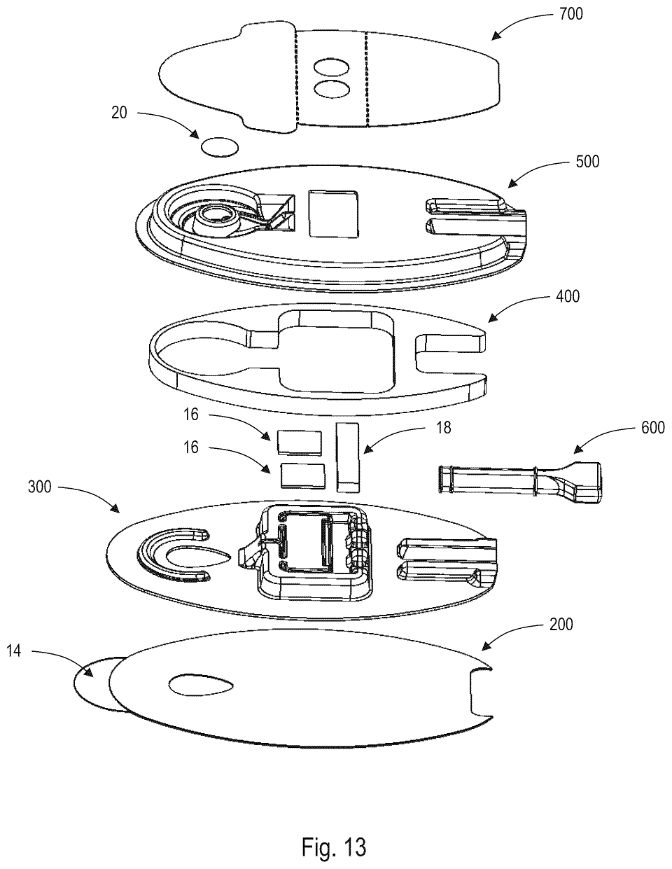

Referring to FIGS. 12 and 13, the cartridge 12 is shown in accordance with an exemplary embodiment. As will be discussed in further detail herein, the cartridge 12 is formed of a plurality of layers of suitable materials including, but not limited to, polymeric materials (e.g., polyolefins, polyethylene terephthalate (PET), polyurethanes, polynorbornenes, polyethers, polyacrylates, polyamides (Polyether block amide also referred to as Pebax®), polysiloxanes, polyether amides, polyether esters, trans-polyisoprenes, polymethyl methacrylates (PMMA), cross-linked trans-polyoctylenes, cross-linked polyethylenes, cross-linked polyisoprenes, cross-linked polycyclooctenes, inorganic-organic hybrid polymers, co-polymer blends with polyethylene and Kraton®, styrene-butadiene co-polymers, urethane-butadiene co-polymers, polycaprolactone or oligo caprolactone co-polymers, polylactic acid (PLLA) or polylactide (PL/DLA) co-polymers, PLLA-polyglycolic acid (PGA) co-polymers, photocross linkable polymers, etc.). In some embodiments, all or a portion of each material layer may be formed of poly(dimethylsiloxane) (PDMS) to allow visibility of components disposed within the cartridge 12. While the various layers of material that form the cartridge 12 are said to be layers of polymeric material, it is understood that the layers of material that form the cartridge 12 may be formed of any suitable material (e.g., metal, metal alloy, etc.). As will be discussed in further detail herein, the layers can be stacked and coupled to one another to form the cartridge 12. In some embodiments, the layers of the cartridge 12 may be formed of aluminum or other suitable metals. In certain embodiments, the layers of the cartridge 12 may be formed of a polymeric coated with aluminum film or another suitable metal film. In some embodiments, the layers of material, including the channels disclosed herein, may be thermoformed.

The cartridge 12 can include an adhesive layer 200, a bottom layer 300 of polymeric material, a desiccant 400, a top layer 500 of polymeric material, and a moveable vacuum pin 600 that is disposed between the bottom layer 300 and the top layer 500. The desiccant can be formed in the space between the top layer 500 and the bottom layer 300. The adhesive layer 200 can be formed of a pressure sensitive adhesive (PSA). The vacuum pin 600 can be thermoformed and laser welded. The vacuum pin can also be made of a polymeric material that is 3D printed or injection molded or compression molded or casting. The cartridge 12 further includes an adhesive cover 700 that covers the top surface of the top layer 500 and a protective liner 14 (e.g., formed of paper) that covers the adhesive layer 200. The protective liner 14 protects the adhesive layer 200 before the cartridge 12 is attached to the skin of a subject. When the adhesive layer 200 is pressed against the user's skin, the PSA can create an airtight seal that can allow the vacuum pin to work more effectively.

As further depicted in FIG. 13, the cartridge 12 also includes a first and second collection pad 16 that are configured to absorb a physiological sample, a hydrophobic foam 18, and a pierceable foil 20 that seals a portion of the cartridge 12. The collection pad 16 can include a DBS/Whatman paper to absorb and collect the physiological sample, as in the CF12 from Cytiva.

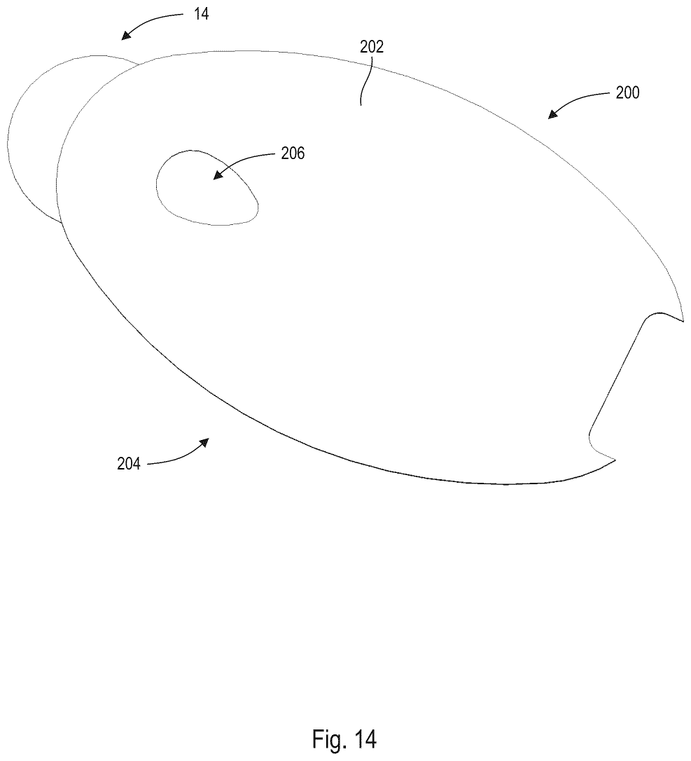

As depicted in FIG. 14, the adhesive layer 200 includes a top surface 202 and an opposed bottom surface 204. The adhesive layer 200 further includes a sample well opening 206 which extends through the adhesive layer 200, but not necessarily through the liner 14. That is, the sample well opening 206 extends between the top surface 202 and the bottom surface 204. FIG. 14 shows the sample well opening having a teardrop shape, but the sample well opening can also have a circular shape in order to reduce the distance between the sample well opening 206 and the needles 110. The adhesive layer 200 has a similar shape and dimension as the bottom layer 300 such that the adhesive layer 200 covers the bottom layer 300. The protective liner 14 covers the adhesive layer 200 and includes a pull tab which allows a user to remove the protective liner 14 from the cartridge 12 thereby allowing a user to attach the cartridge 12 to the skin of a subject.

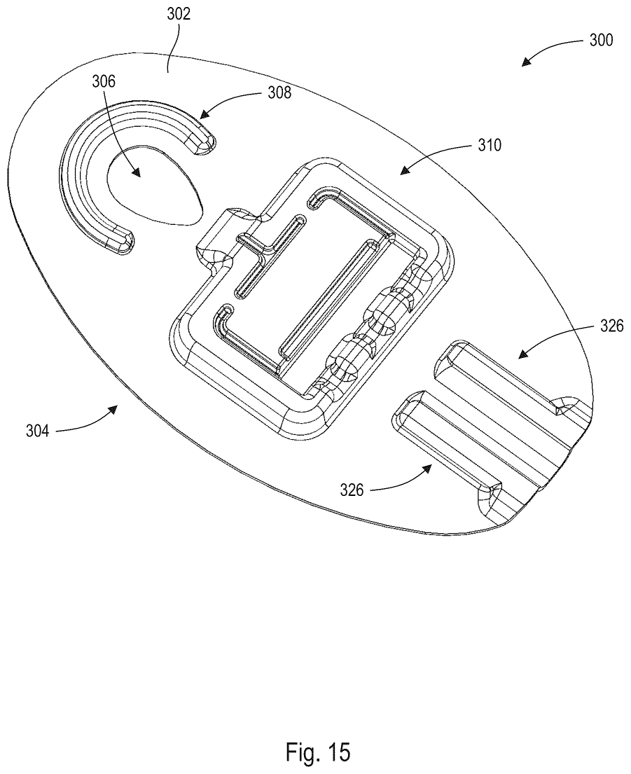

The bottom layer 300 (FIG. 15) can have a thickness in a range of about 0.4 mm to about 0.6 mm (e.g., 0.5 mm) and includes a top surface 302 and an opposed bottom surface 304. The bottom layer 300 also includes a sample well opening 306 that extends through the bottom layer 300. That is, the sample well opening 306 extends between the top surface 302 and the bottom surface 304. The bottom layer 300 further includes a rim 308 that extends substantially vertically from and perpendicular to the top surface 302. The rim 308 at least partially surrounds the sample well opening 306. As will be discussed in further detail herein, the rim 308 aids in securing the bottom layer 300 to the top layer 500.

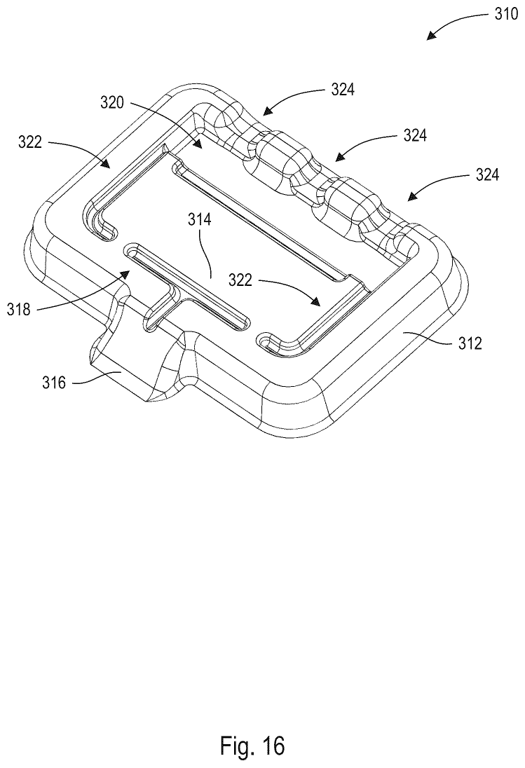

The bottom layer 300 further includes a collection pad support 310 (FIG. 16). The collection pad support is generally rectangular and includes a side wall 312 and a top wall 314. The side wall 312 extends substantially vertically from and perpendicular to the top surface 302. The top wall 314 extends substantially horizontally from and between the side wall 312. The collection pad support 310 further includes a slanted wall 316 that extends between the top surface 302 and the top wall 314. The top wall 314 of the collection pad support 310 includes a T-shaped groove 318. As will be discussed in further detail herein, a physiological sample drawn from a subject travels along a portion of the slanted wall 316 to the T-shaped groove 318. The top wall 314 further includes a hydrophobic foam receptacle 320, opposing side grooves 322, and three notches 324. The side grooves 322 and the three notches 324 are in open communication with the hydrophobic foam receptacle 320 that retains the hydrophobic foam 18. The bottom layer 300 further includes two vacuum pin supports 326 (FIG. 15) that extend substantially vertically from and perpendicular to the top surface 302. The vacuum pin supports 326 are shaped to accept at least a portion of the vacuum pin 600.

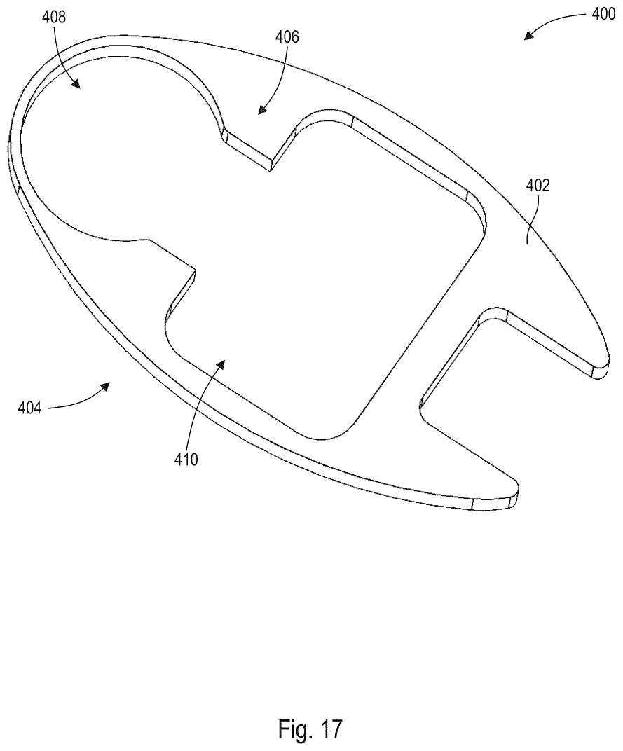

Referring now to FIG. 17, the desiccant 400 is shown in accordance with an exemplary embodiment. The desiccant 400 can have a thickness of about 3.5 mm and includes a top surface 402 and an opposed bottom surface 404. The desiccant 400 includes an opening 406 that extends through the desiccant 400. That is, the opening 406 extends between the top surface 402 and the bottom surface 404. The opening 402 includes a rounded portion 408 and a rectangular portion 410. When the cartridge 12 is assembled, the bottom layer 300 and the top layer 500 define an inner void of the cartridge 12 that is shaped to accept and accommodate the desiccant 400. Furthermore, the rounded portion 408 of the opening 402 has a similar shape and dimension as the rim 308 and the rectangular portion 410 of the opening 402 has a similar shape and dimension as the side wall 312 of the collection pad support 310 such that desiccant 400 contacts the rim 308 and the collection pad support 310. This contact prevents the desiccant 400 from moving within the cartridge 12. The desiccant 400 also includes an indentation 412 (show in FIG. 17). The indentation 412 is formed such that the desiccant 400 does not contact the vacuum pin supports 326 when the cartridge 12 is assembled. The desiccant 400 is formed of a desiccant material (e.g., silica gel, calcium chloride, molecular sieve, activated carbon, activated alumina, aluminum oxide, moisture absorbing polymers, desiccant-infused absorbent pads, etc.). As such, the desiccant 400 dries substances that are in open communication with it. As will be discussed in further detail herein, the desiccant 400 dries a physiological sample that has been absorbed by a collection pad 16 of the cartridge 12.

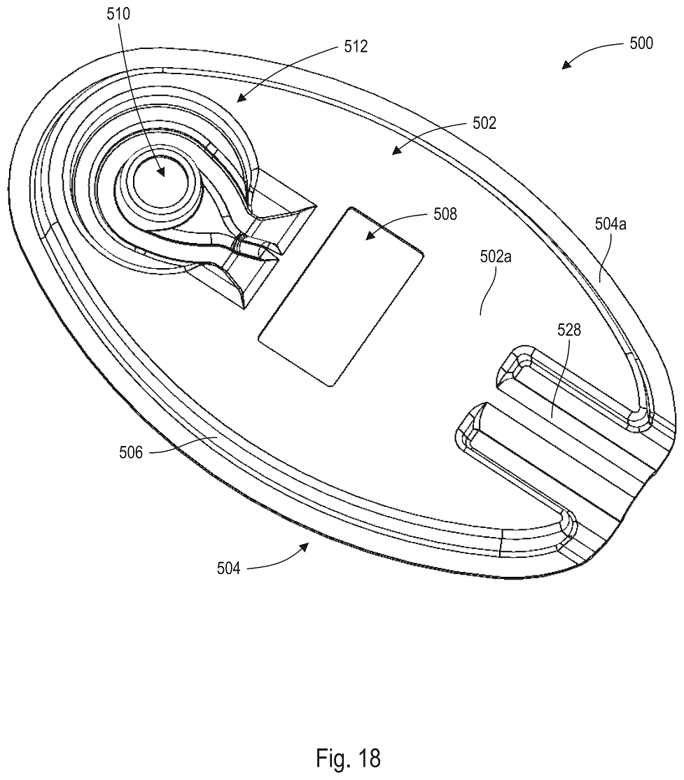

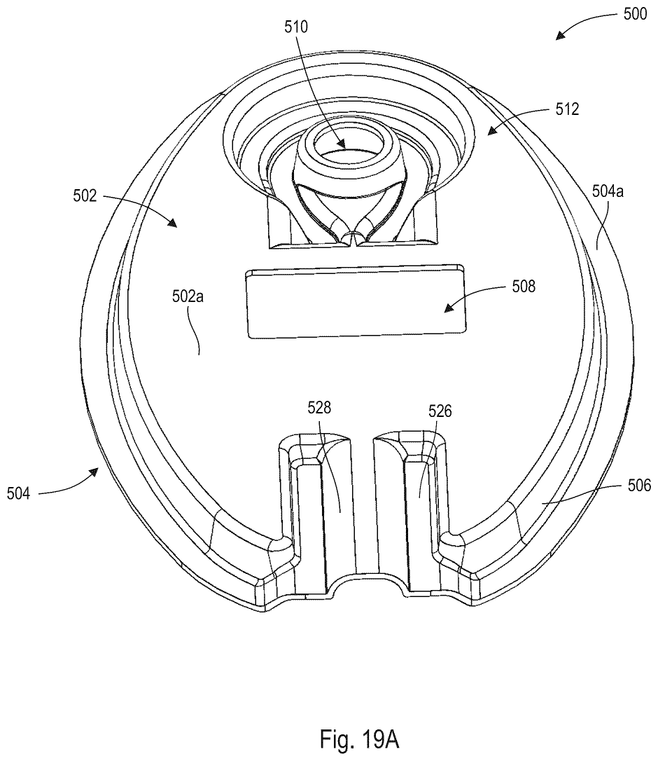

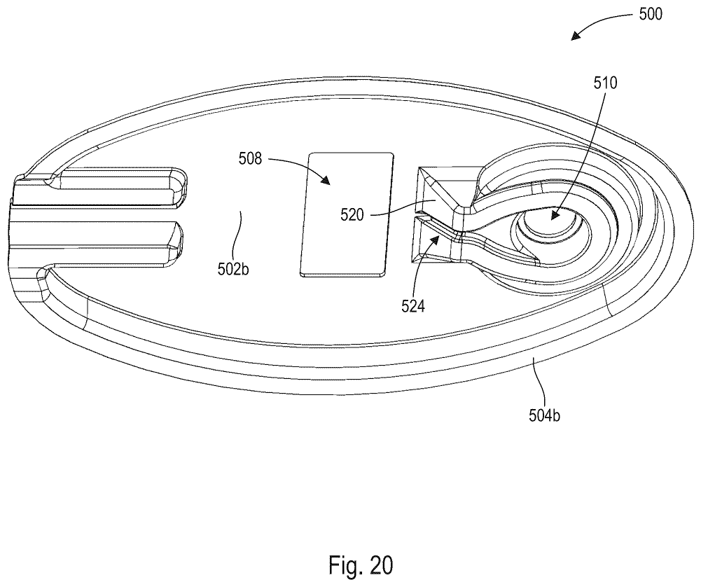

With reference to FIGS. 18-20, the top layer 500 is shown in accordance with an exemplary embodiment. The top layer 500 can have a thickness in a range of about 0.25 mm to about 3.0 mm (e.g., 0.75 mm) and includes a top wall 502, a bottom wall 504, and a side wall 506 that extends between the top wall 502 and the bottom wall 504. The top wall 502 includes an outer surface 502a and an opposed inner surface 502b. The bottom wall 504 includes a top surface 504a and an opposed bottom surface 504b. The side wall 506 extends substantially vertically from and perpendicular between the top wall 502 and the bottom wall 504. When the cartridge 12 is assembled, the bottom surface 504b of the bottom wall 504 is affixed to the top surface 302 of the bottom layer 300. The top layer 500 may be affixed to the bottom layer 300 by laser welding, heat sealing, thermal bonding, pressure sensitive adhesive, heat induction, adhesive bonding, ultrasonic welding diffusion bonding, or mechanical locking. Furthermore, the top wall 502, the side wall 504, and the top surface 302 of the bottom layer 300 define an inner volume of the cartridge 12 in which the desiccant 400 is disposed.

The top layer 500 further includes a sample collection pad opening 508 that extends through the top wall 502. That is, the opening 508 extends between the outer surface 502a and the inner surface 502b of the top wall 502. As will be discussed in further detail herein, the sample collection pad opening 508 is shaped and dimensioned to accommodate two sample collection pads 16. The top layer 500 also includes a needle aperture 510 that extends through the bottom wall 504. That is, the needle aperture 510 extends between the top surface 504a and the bottom surface 504b of the bottom wall 504. When the cartridge 12 is assembled, the needle aperture 510 is aligned with the sample well opening 306. The needle aperture 510 can be made smaller than the sample well opening 206 in order to hold the skin closer to the lancet, thereby helping prevent the skin from excessive tenting caused by blade penetration.

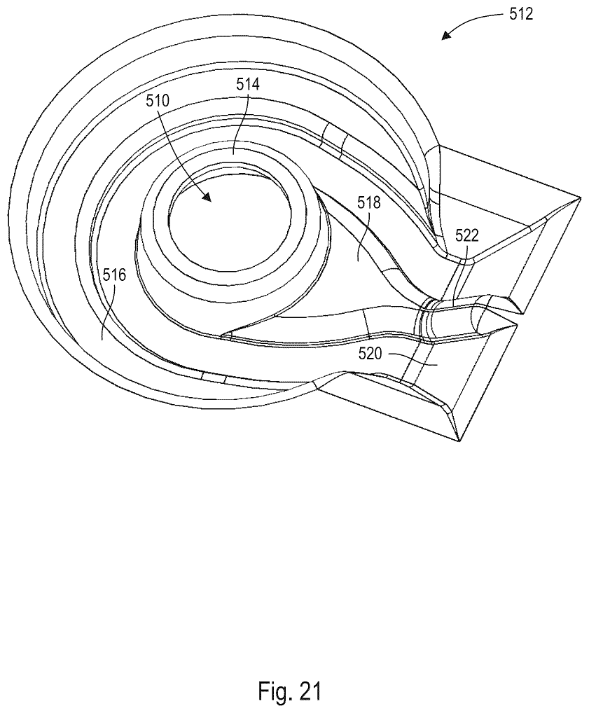

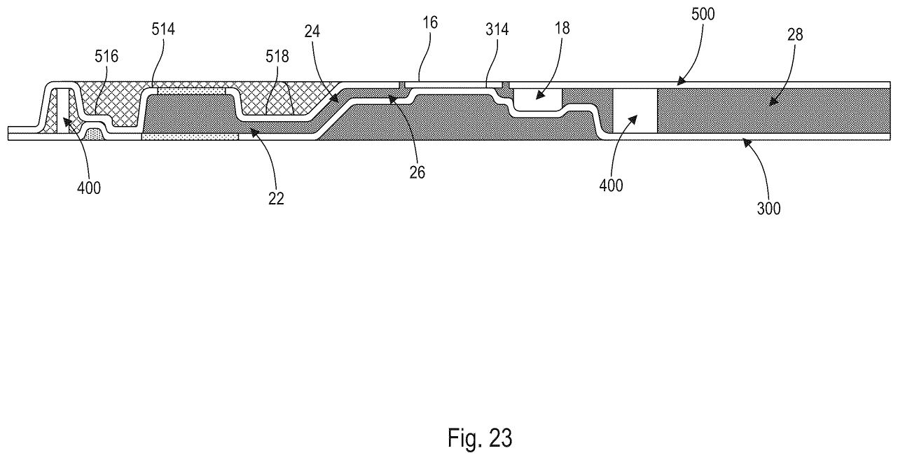

The top layer 500 further includes a lancet receiving element 512 (FIG. 21) that extends about the needle aperture 510. The lancet receiving element 512 includes a circular extension 514 that extends vertically from and perpendicular to the top surface 504a of the bottom wall 504. The circular extension 514 surrounds and is substantially concentric with the needle aperture 510. As will be discussed in further detail herein, the circular extension 514 actuates the lancet 100 to draw a physiological sample from a subject. The lancet receiving element 512 further includes a rounded wall 516 that extends substantially vertically from and perpendicular to the top surface 504a of the bottom wall 504. The lancet receiving element 512 further includes a sample well wall 518 that extends substantially vertically from and perpendicular to the top surface 504a of the bottom wall 504. The sample well wall 518 also extends substantially horizontally form and perpendicular to the circular extension 514. When the cartridge 12 is assembled, the sample well wall 518 and the sample well opening 306 define a physiological sample well 22 (FIG. 23) that is in open communication with the needle aperture 510.

The top layer 500 further includes a slanted wall 520 that extends between the top wall 502 and the bottom wall 504. The slanted wall 520 includes a semicircular extension 522 that defines a groove 524 of the slanted wall 520. The slanted wall 316 and the slanted wall 520 have substantially the same slope such that when the cartridge 12 is assembled, the slanted walls 520 and 316 contact and are substantially parallel to one another. The groove 524 and the slanted wall 316 define a first channel 24 of the cartridge 12 (FIG. 23). The first channel 24 extends from and is in open communication with the sample well 22. Furthermore, the T-shaped groove 318 and the top wall 502 define a second channel 26 that is in open communication with the first channel 24. As will be discussed in further detail herein, the first channel 24 and the second channel 26 carry a physiological sample from the sample well 22 to the collection pads 16. The first and second channels 24 and 26 can have a diameter in the range of approximately 0.5 mm-1.5 mm.

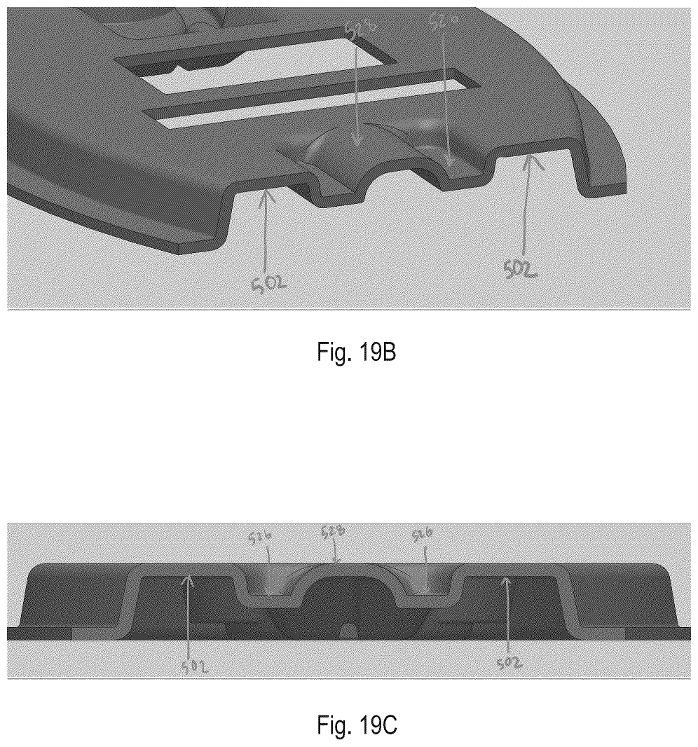

The top layer 500 also includes a U-shaped wall 528 and flat sections 526 adjacent the U-shaped wall 528 (see FIGS. 19A-19C). The edge portions of the U-shaped wall 528 extend substantially vertically from the bottom wall 502 and the flat sections 526 extend vertically from the flat sections 526. The U-shaped wall 528 has a similar shape and dimension as the vacuum pin supports 326 such that when the cartridge 12 is assembled, the vacuum pin supports 326 contact the U-shaped wall 528. The U-shaped wall 528 and the vacuum pin supports 326 define a vacuum pin receptacle 28 (FIG. 23) that is shaped and dimensioned to accept a portion of the vacuum pin 600.

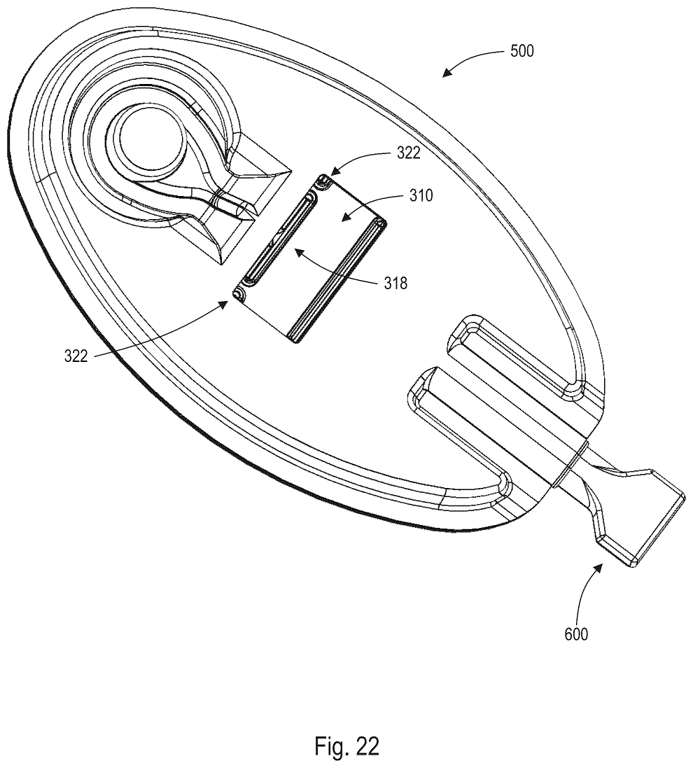

As depicted in FIG. 22, when the cartridge 12 is assembled, the collection pad support 310 is aligned with the sample collection pad opening 508 such that the collection pads 16 disposed on the top wall 314 of the collection pad support 310 extend through the sample collection pad opening 508. The collection pads 16 are positioned on the collection pad support 310 such that the collection pads 16 are positioned above a portion of the T-shaped groove 318 and a portion of a side groove 322.

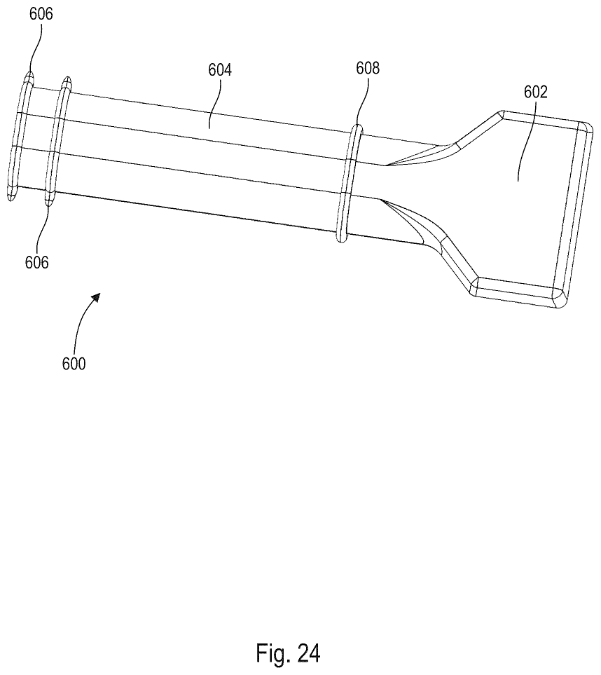

Referring now to FIG. 24, the vacuum pin 600 is shown in accordance with an exemplary embodiment. In this embodiment, the vacuum pin 600 includes a handle 602 and a neck 604 that extends from the handle 602. The vacuum pin 600 also includes a plurality of ridges 606 that extend vertically from and around the neck 604. In some embodiments, the ridges 606 may be formed of an elastomeric material. The vacuum pin 600 also includes a rim 608 that extends substantially vertically from and around the neck 604. The rim 608 has a wider diameter than the ridges 606 and prevents a user from pushing the vacuum pin 600 farther into the cartridge 12 as the rim 608 contacts the bottom layer 200 and the top layer 500 when pushed. When the vacuum pin 600 is disposed within the cartridge 12, the ridges 606 contact the surfaces of the vacuum pin receptacle 28 (e.g., the bottom surface 302, the vacuum pin supports 326, and the U-shaped wall 528) thereby providing an airtight seal between the vacuum pin 600 and the vacuum pin receptacle 28.

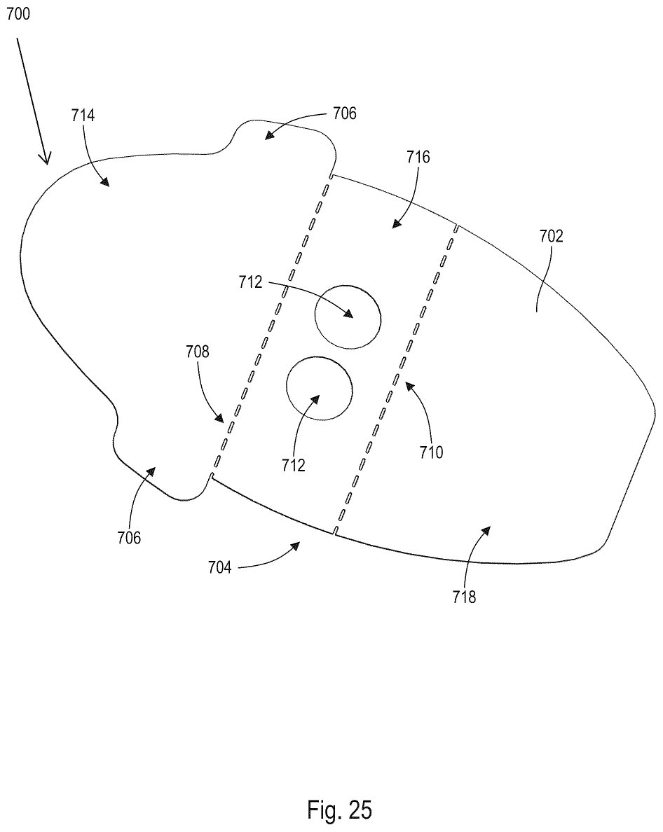

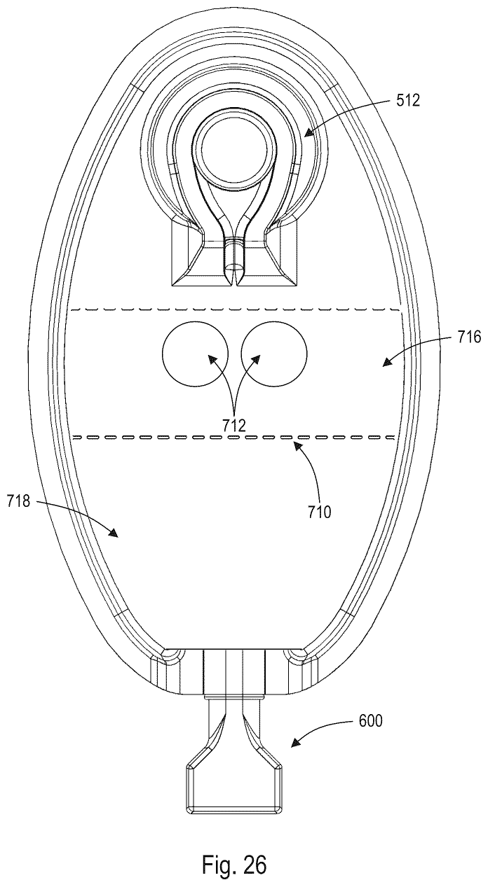

With reference to FIG. 25, the adhesive cover 700 includes a top surface 702, an opposed bottom surface 704, pull tabs 706, a first perforation 708, a second perforation 710, and viewing apertures 712. The bottom surface 704 includes an adhesive that affixes the bottom surface 704 to the top wall 502 of the top layer 500. The viewing apertures 712 are aligned with the collection pads 16 and may be covered with a transparent film that allows a user of the dermal patch system 10 to view the collection pads 16 in order to visually confirm blood collection. As depicted in FIG. 26, user can pull a pull tab 706 to separate first portion 714 of the adhesive cover 700 from a second portion 716 and a third portion 718 of the adhesive cover 700 by ripping the adhesive cover 700 at the first perforation 708 to expose the lancet receiving element 512. A medical professional can further rip the adhesive cover 700 at the second perforation 710 to remove the second portion 716 from the third portion 718 of the adhesive cover 700 to expose the collection pads 16. Serial numbers as well as QR codes can be printed on the adhesive cover 700 to identify the cartridge in order to preserve the chain of custody.

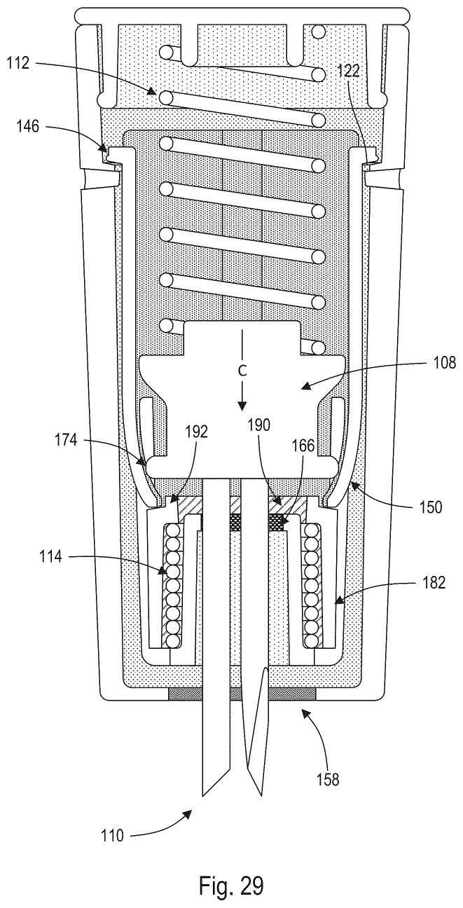

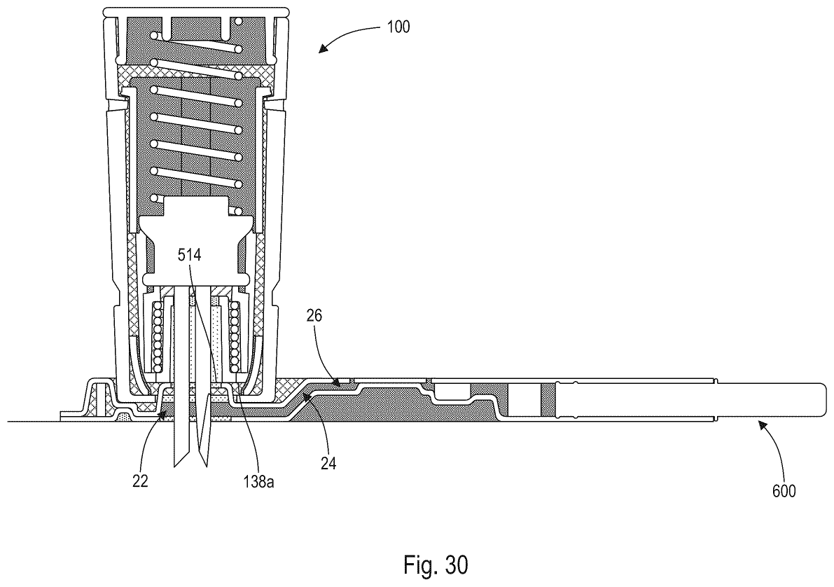

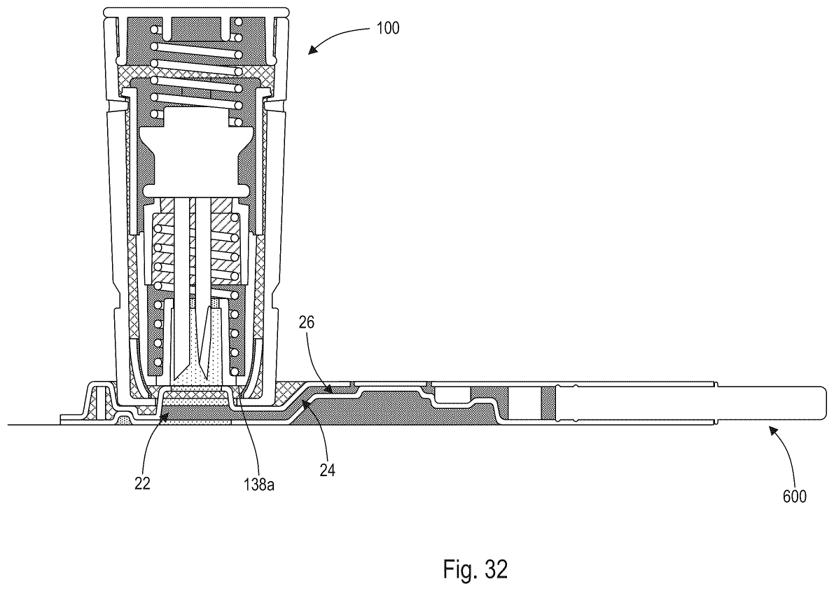

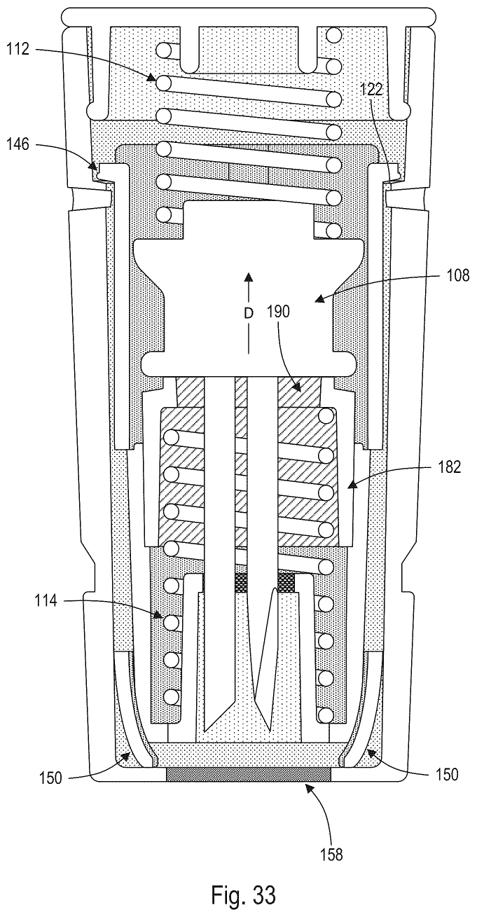

The lancet 100 is moveable between an undeployed position (FIG. 1), a deployed position (FIG. 29), and a retracted position (FIG. 32).

In the undeployed position, the hooks 146 of the inner sleeve 106 extend through the openings 126 of the housing 102 which retains the lancet 100 in the undeployed position and prevents the lancet 100 from moving to the deployed position. In this position, the extensions 144 are compressed inward toward the center of the lancet 100 which allows the needle platform 108 to rest upon the inner sleeve 106 which prevents the needle platform from moving vertically downward and into the deployed position. Specifically, the angled surface 178 of the extensions 176 of the needle platform 108 contacts rests upon the extensions 144.

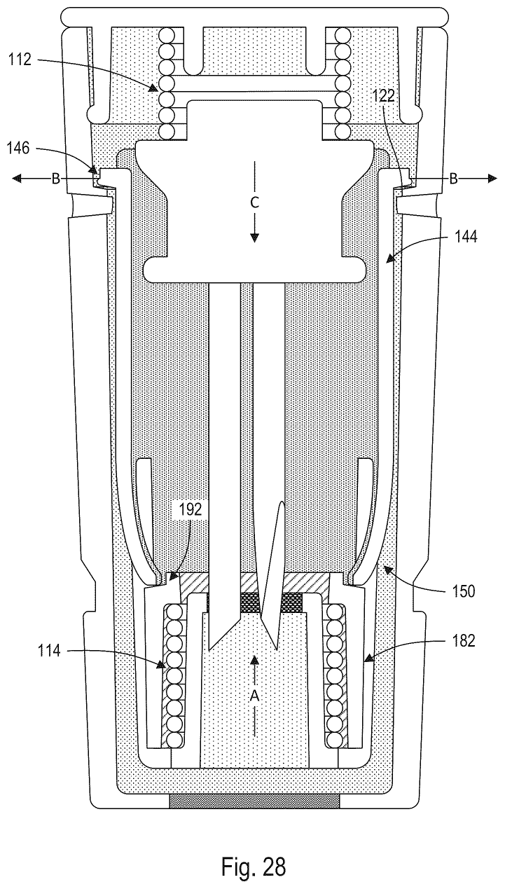

Furthermore, the injection spring 112 extends around the body 170 of the needle platform 108 and the outer surface 136a of the inner cylinder 136. The injection spring 112 extends substantially vertically between the inner surface 132b of the top wall 132 of the cap 104 and the upper circular extension 172 of the needle platform 108. In the undeployed position, the injection spring 112 is compressed between the top wall 132 of the cap 104 and the upper circular extension 172 of the needle platform 108. The retraction spring 114 extends around the post 160 of the inner sleeve 106. The retraction spring 114 extends substantially vertically between the bottom wall 140 of the inner sleeve 106 and the top wall 186 of the spring sleeve 182. That is, the retraction spring 114 extends substantially vertically between the inner surface 140b of the bottom wall 140 of the inner sleeve 106 and the inner surface 186b of the top wall 186 of the spring sleeve 182. When the lancet 100 is in the undeployed position, the retraction spring 114 is compressed between the bottom wall 140 of the inner sleeve 106 and the top wall 186 of the spring sleeve 182, as shown in FIG. 28. Furthermore, in the undeployed position, the deformable tabs 150 contact the ledge 192 of the spring sleeve 182 which prevents the retraction spring from expanding and moving the spring sleeve 182.

After a user removes the first portion 714 of the adhesive cover 700 to expose the lancet receiving element 512, the lancet 100 can be engaged with the cartridge 12 by pushing the lancet 100 into the lancet receiving element 512. This causes the needles 110 of the lancet 100 to automatically move from an undeployed position to a deployed position and automatically from the deployed position to a retracted position.

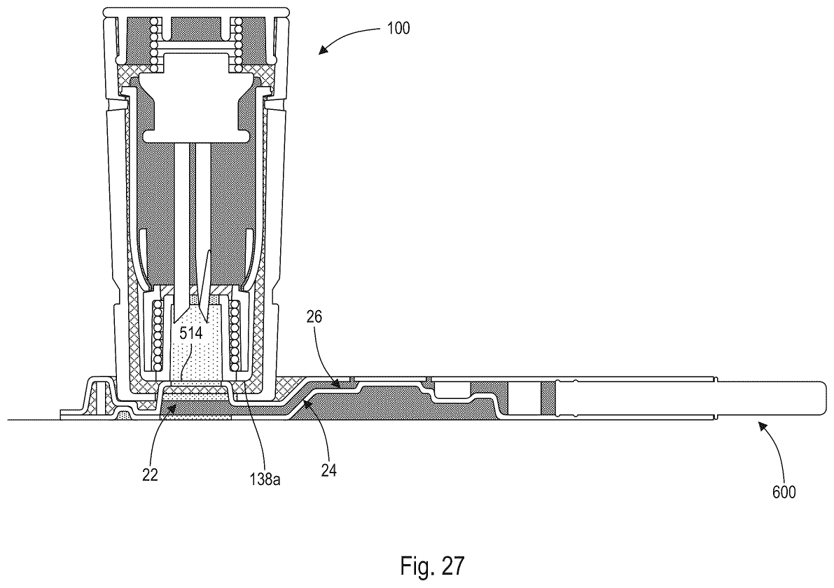

As depicted in FIGS. 27, 29, and 31, when the lancet 100 engages with the cartridge 12, the bottom wall 140 rests upon the rounded wall 516 and the sample well wall 518 (see FIG. 21). Furthermore, upon engagement, the circular extension 514 of the lancet receiving element 512 extends through the aperture 128 of the housing 102 and contacts the bottom wall 140 of the inner sleeve 106. Specifically, the top surface of the circular extension 514 contacts the outer surface 140a of bottom wall 140 which forces the inner sleeve 106 to move vertically upward in the direction of arrow A within the housing 102. That is, the movement of the inner sleeve 106 is activated automatically when the profile of the lancet 100 (i.e., the bottom wall 120 of the lancet 100) interfaces with the profile of the cartridge 12 (i.e., circular extensions 514 of the cartridge 12). As previously discussed herein, grooves 124 of the housing 102 and the protrusions 154 of the inner sleeve 106 guide the vertical movement of the inner sleeve 106. The vertical movement of the inner sleeve 106 causes the hooks 146 to disengage from the openings 126 and causes the extensions 144 to extend vertically above and rest upon the ledge 122. In his position, the extensions 144 decompress and expand outwardly in the direction of arrow B.

When the extensions 144 expand, the needle platform 108 no longer rests upon the extensions 144 which allows the needle platform 108 to move vertically downward in the direction of arrow C. As the needle platform 108 moves downward, the injection spring 112 is allowed to expand and the force applied by the injection spring 112 causes the needle platform 108 (and therefore the needles 110) to travel with a force that is sufficient to cause the needles 110 to puncture the skin of a subject wearing the dermal patch system 10. The injection spring 112 causes the needles 110 to pierce the foil 20 and to extend through the spring sleeve 182 via the opening 190, through the post 160 via the opening 166, through the opening 158 of the inner sleeve 106 and exit the housing 102 via the aperture 128 and pass through the cartridge 12 via the well opening 306 and the needle aperture 510. Stated another way, the injection spring 112 moves the needles 110 to the deployed position. Accordingly, the lancet 100 can only be actuated when the profile/configuration of the lancet 100 engages with a matching profile/configuration of the cartridge 12. Therefore, the lancet 100 cannot be actuated by simply pressing the lancet against the skin, thereby preventing unsafe deployment of the needles in the lancet 100.

The parameters of the injection spring 112 can be selected to create the desired injection velocity, penetration force and penetration depth. The parameters of the retraction spring 114 can be selected to create the desired retraction depth within the lancet when the needles are fully retracted to meet desired safety standards (e.g., ISO standards). For example, the force of the injection spring 112 can be set in the range of approximately 13N to approximately 18N. Using an injection spring with these spring forces can create an injection velocity range of approximately 10 m/s to approximately 13 m/s. The force of the retraction spring 114 can be set in the range of approximately 25 N to approximately 32 N, preferably at 28N. Using a retraction spring with these spring forces can create a minimum retraction depth of approximately 3.5 mm to approximately 1.5 mm.

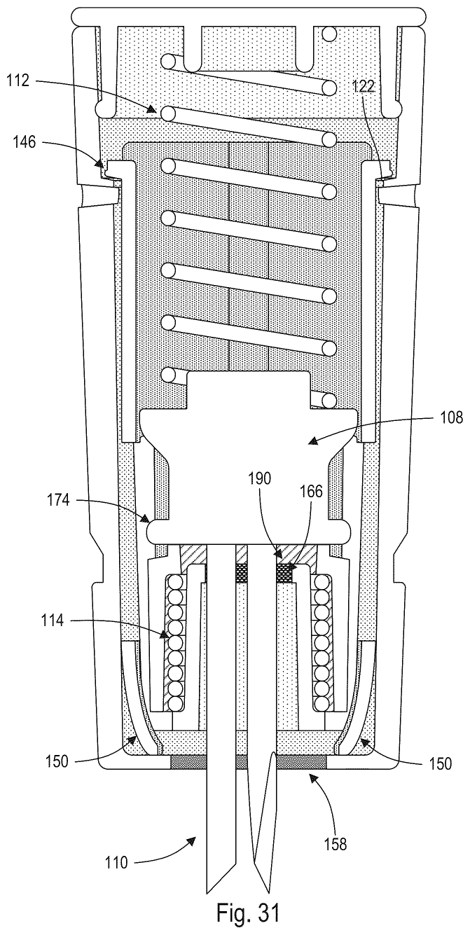

As previously discussed herein, the grooves 156 of the inner sleeve 106 and the projections 180 of the needle platform 108 guide the vertical movement of the needle platform 108. As the needle platform moves in the direction of arrow C, the lower circular extension 174 contacts the deformable tabs 150 (FIG. 31). This contact causes the tabs 150 to deflect away from the spring sleeve 182 of the lancet 100 or causes the tabs 150 to break. As depicted in FIG. 32, when the tabs 150 break, the tabs 150 fall into a void between the inner sleeve 106 and the housing 102. In the deployed position, the needles 110 extend beyond the housing 102 and the lower circular extension 174 contacts the top wall 186 of the spring sleeve 182. Once the tabs 150 break, the lancet cannot be used again (i.e., single use). This is because once the retraction spring 114 is released, the blades are held back and are no longer set in the correct position.

When the tabs 150 deflect away from the spring sleeve 182 or break, the tabs 150 no longer contact the ledge 192 of the spring sleeve 182. As a result, the retraction spring 114 is allowed to expand. The expansion of the retraction spring 114 moves the spring sleeve 182 vertically upward in the direction of arrow D. If the tabs 150 deflect and do not break, the spring sleeve 182 prevents the tabs 150 from contacting the retraction spring 114 as the retraction spring 114 expands. The spring sleeve 182 moves the needle platform 108 in the direction of arrow D to place the lancet 100 in the retracted position. That is, after moving to the deployed position, the retraction spring 114 causes the needles 110 to retract back into the inner volume 142 of the inner sleeve 106 the well opening 306 of the bottom layer 300, the needle aperture 510 of the top layer 500, the aperture 128 of the housing 102, and the opening 158 of the inner sleeve 106. That is, after penetrating the skin of a subject, the retraction spring 114 causes the needles 110 to automatically retract back into the housing of the lancet 100 thereby placing the lancet 100 in the retracted position.

By simultaneously moving three needles 110 to the deployed position, the lancet 100 punctures the skin of the subject at three locations at a same time. The three needles cause the tensioning of the skin such that one of the punctures created by the needles 110 penetrates the skin at a different depth. As a result, it is possible to have a higher volume of blood flow out of one of the punctures relative to the other two. This can occur when some of the needles tent slightly more such that the other needle(s) puncture a portion of the skin that is slightly more tensioned. That is, the three needles 110 are positioned on the needle platform 108 such that one of the needles 110 draws a majority of the physiological sample. Also, using three needles increases the chances of penetrating a capillary and puncturing the skin, thereby resulting in a more effective blood draw. The spring sleeve can help with the alignment of the needles. Also, the orientation of the needles can be selected to increase the distance between puncture points. For example, when the blade tips are pointed outward, the deepest points of puncture are further away from each other compared to when the blade tips are pointed inwards. Having a greater distance between puncture points can increase the likelihood that a capillary is targeted, thereby increasing the probability of bleeding. However, when the blade tips are pointed outward, less skin can be pinched, which can reduce the amount of bleeding. Overall, it has been found that there is better bleeding when the blade tips are pointed outward.

The needles 110 can be coated with silicone to minimize friction and tenting of the skin, which helps prevent the needles from sticking to the skin, thereby helping to minimize injection pain. Specifically, Silbione™ medical grade silicone fluids, such as Elkem and Nusil, can be used. This can also reduce break-loose force and insertion and drag forces. Also, the geometry of the blades of the needles 110 can be selected to promote bleeding. The blade tip geometry variables that impact the width and diameter of skin penetration include bevel length, bevel angle, tip angle and blade diameter. The blade diameter can be selected to be in the range of approximately 1.5 mm to 1.69 mm, preferably 1.60 to 1.64 mm, for example 1.62 mm, as shown in FIG. 42. The tip angle can be selected to be in the range of approximately 15 degrees to 75 degrees, preferably 45 degrees+/=5 degrees. The bevel length can be selected to be in the range of approximately 3 mm to 8 mm, for example 4.5 mm+/−0.5 mm. Also, the bevel angle can be selected to be in the range of approximately 15 degrees to 75 degrees, for example 35 degrees. These blade dimensions can be selected to increase the cross sectional surface area of the blade 4 mm from the tip of the blade, as shown in FIG. 42, which can create a wider wound and result in more bleeding.

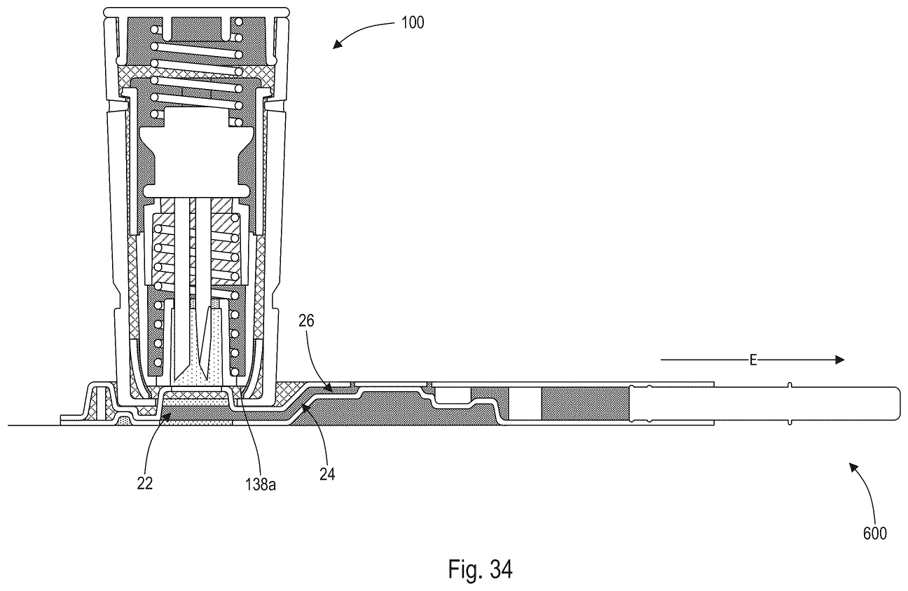

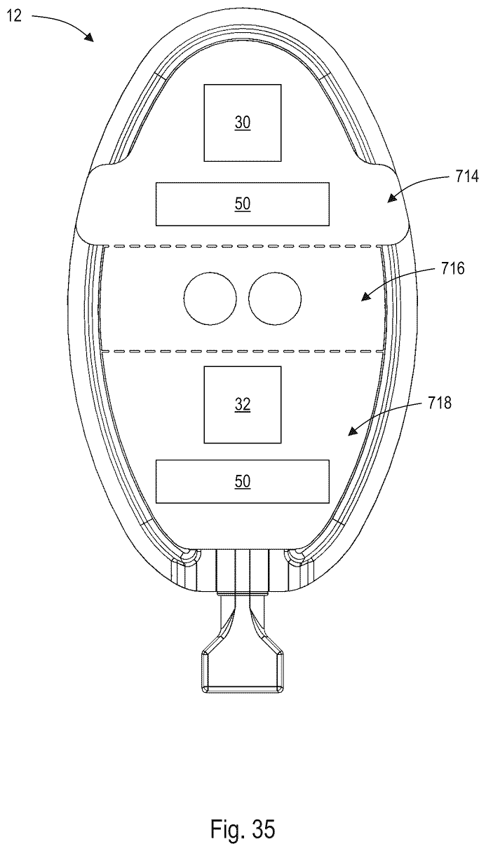

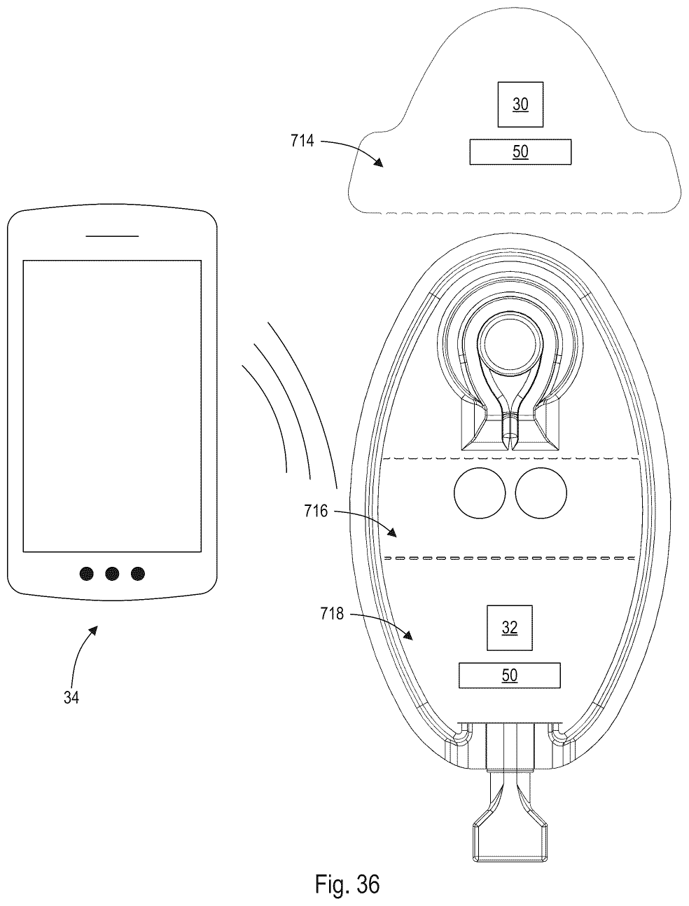

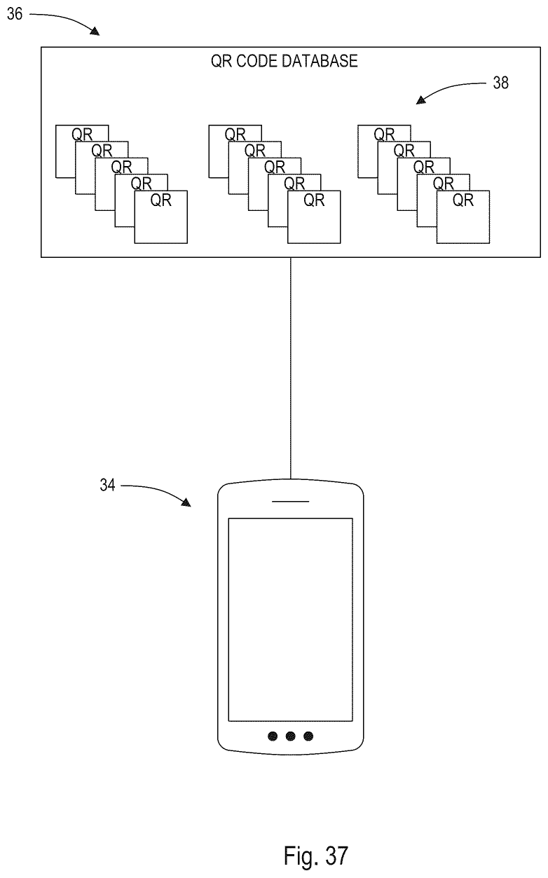

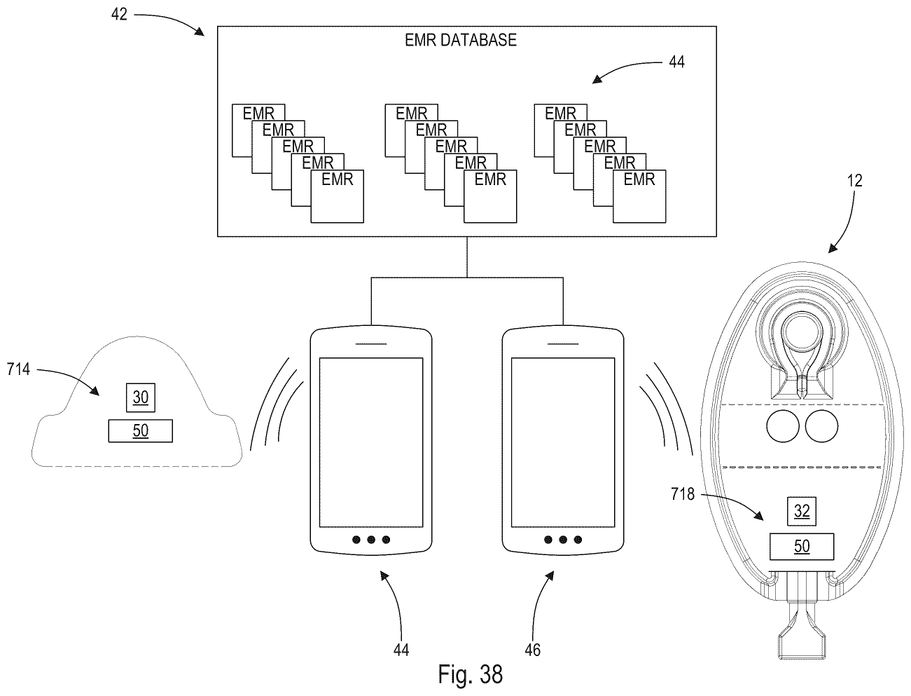

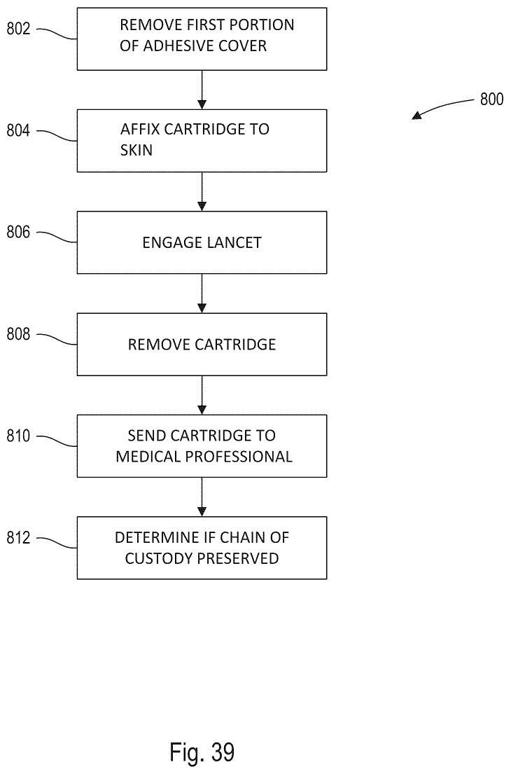

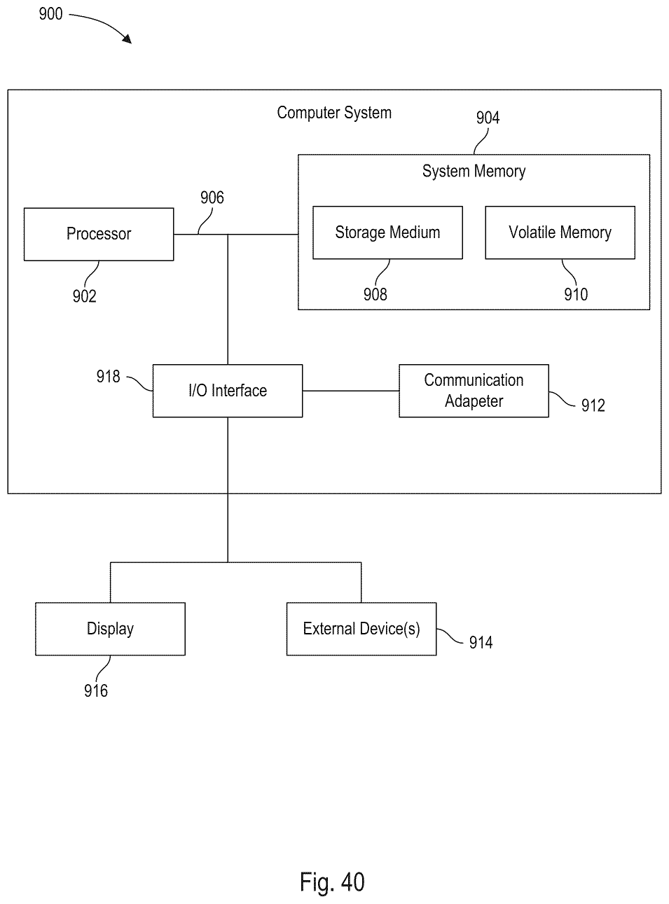

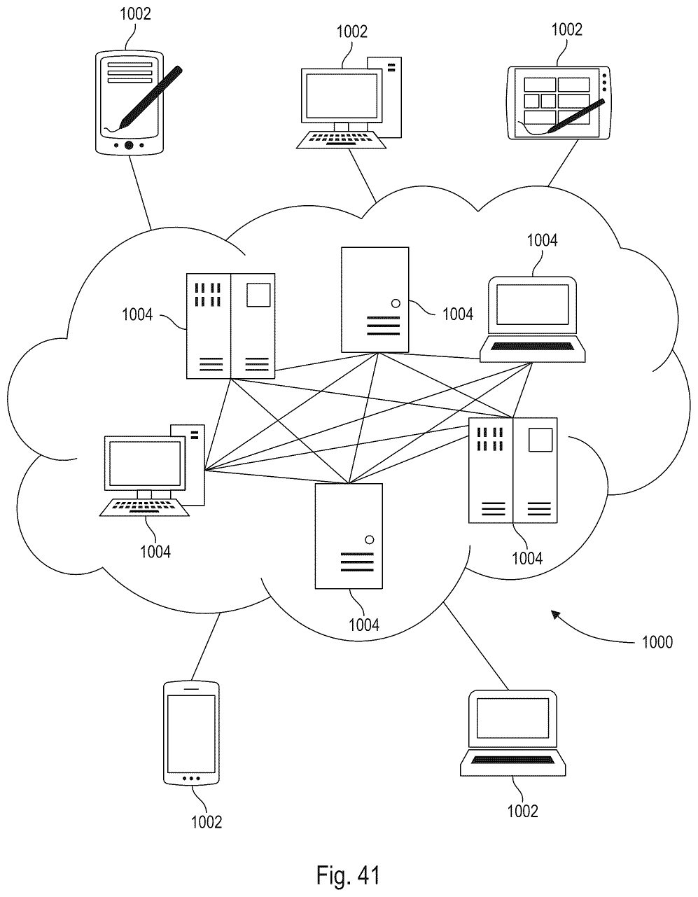

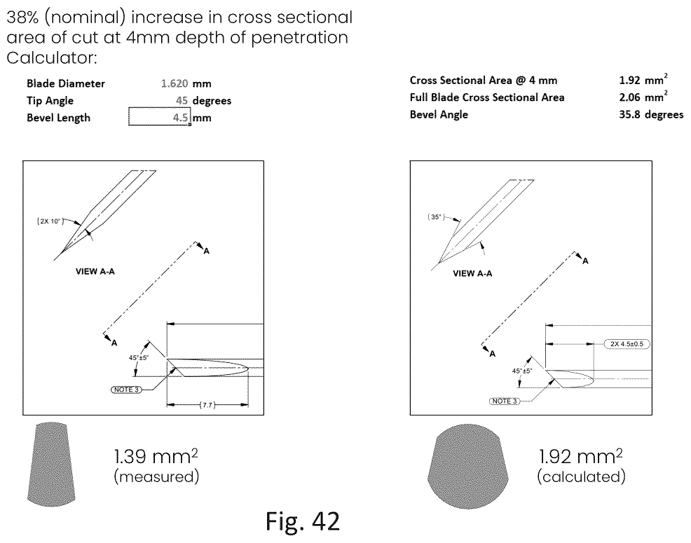

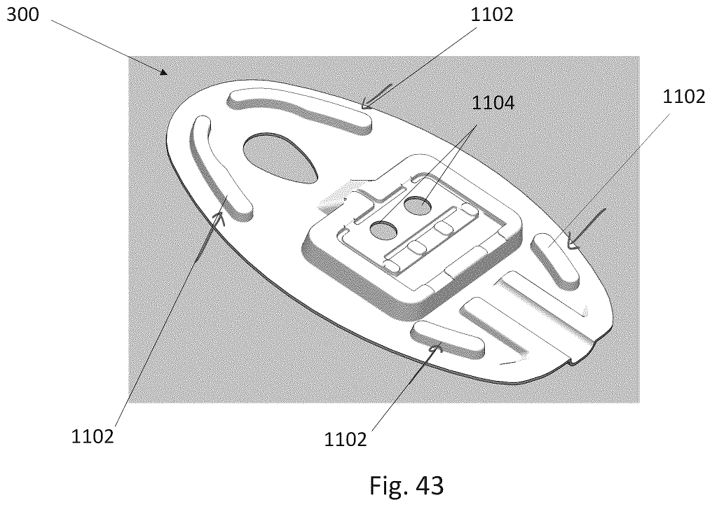

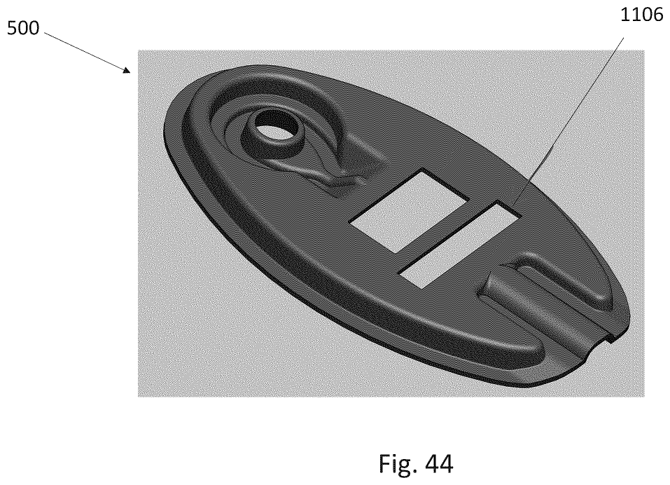

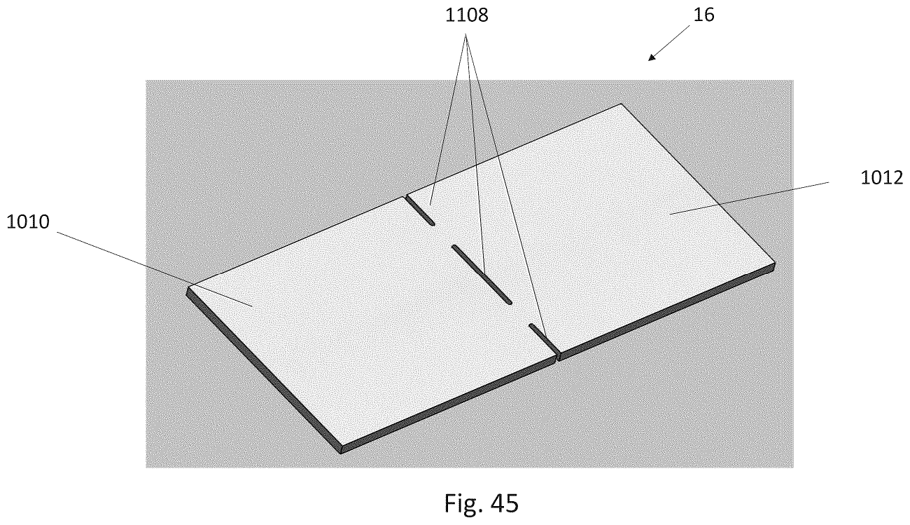

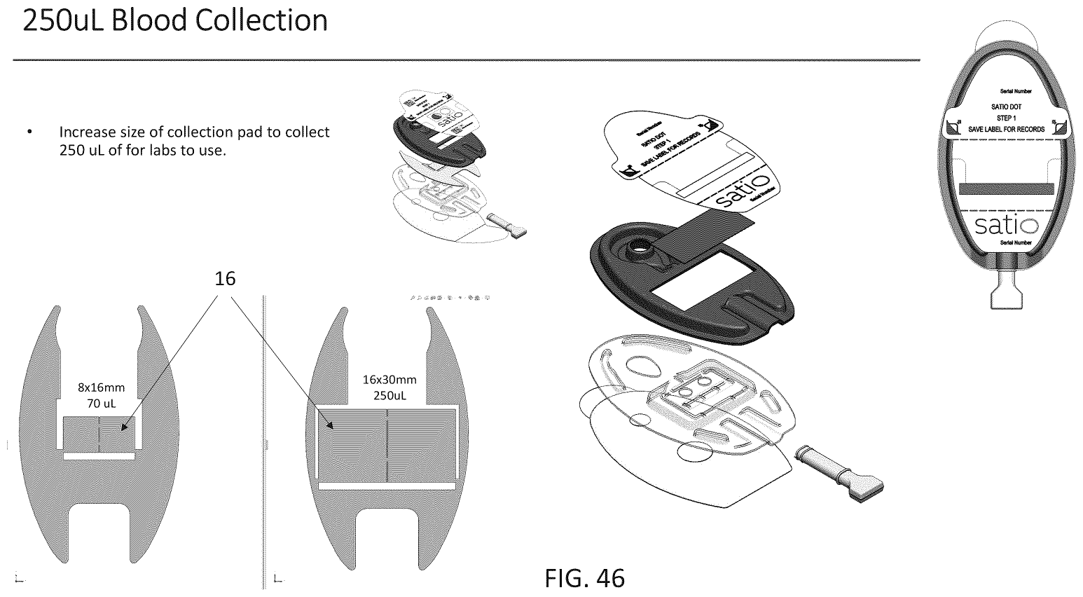

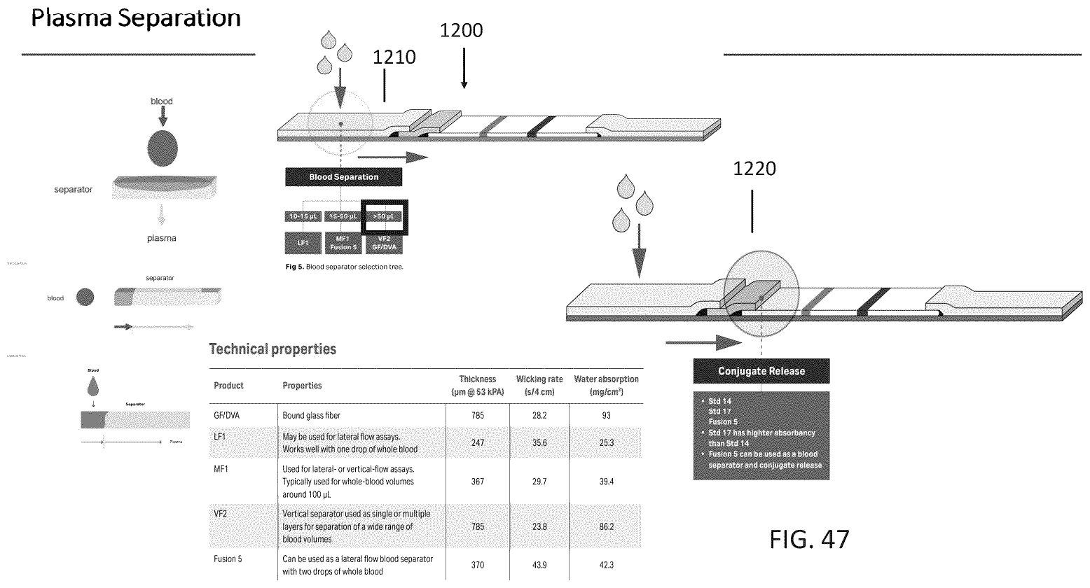

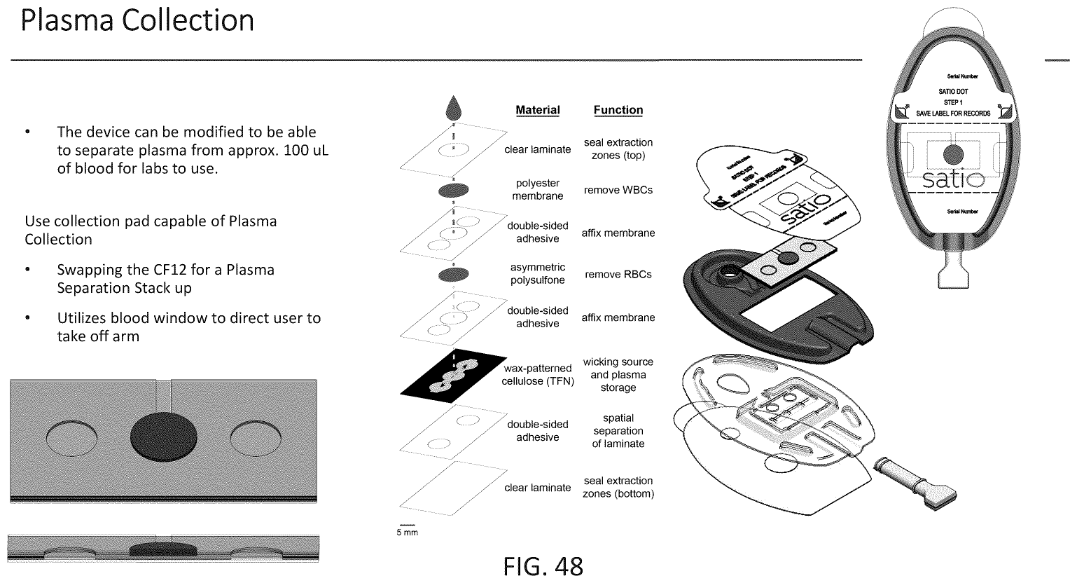

After the needles 110 retract, a physiological sample pools within the physiological sample well 22 of the cartridge 12. When the physiological sample is within the physiological sample well 22, a user can pull the vacuum pin 600 in the direction of arrow E (FIG. 34) to move the vacuum pin 600 from a first position (also referred to as an “undeployed” position) to a second position (also referred to as an “deployed” position). Moving the vacuum pin 600 to the deployed position creates a vacuum the cartridge 12. More specifically, since the desiccant 400 is formed of a porous material, moving the vacuum pin 600 to the deployed position creates a vacuum within the first channel 24 and the second channel 26 via the grooves 324 of the collection pad support 310. The strength of the vacuum is proportional to the amount the vacuum pin 600 moves. That is, the more the vacuum pin 600 moves, the stronger the vacuum. This vacuum causes the drawn physiological sample to travel to the collection pads 16 via the first channel 24 and the second channel 26. In some embodiments, capillary flow, wicking and gravity assist the vacuum pin 600 in drawing the physiological sample towards the collection pads 16. The collection pads 16 absorb the drawn physiological sample. The user can determine the collection pads 16 have absorbed the physiological sample by viewing the collection pads 16 through the viewing apertures 712 of the adhesive cover 700. Furthermore, the hydrophobic foam 18 prevents physiological sample from exiting the collection pads 16 as the hydrophobic foam 18 repels physiological sample back towards the collection pads 16. The desiccant material of the desiccant 400 then dries the physiological sample on the collection pads 16 via the side grooves 322 side grooves 322.

After the collection pads 16 have absorbed the physiological sample, the user can remove the cartridge 12 from the subject's skin. The user can then send the cartridge 12 to a laboratory where a medical professional can expose the collection pads 16 by removing the second portion 716 from the third portion 718 of the adhesive cover 700 as previously discussed herein. Once removed, the medical professional can apply various solutions to the collection pads 16 which mixes with the physiological sample to form a processed physiological sample that has been freed from the collection pads 16. The processed physiological can then be analyzed.

With reference to FIG. 35 and FIG. 36, in some embodiments, the adhesive cover 700 includes a first quick response (“QR”) code 30 disposed on the first portion 714 of the adhesive cover 700 and a second QR code 32 disposed on the third portion 718 of the adhesive cover 700. In one embodiment, the QR codes 30 and 32 can be used to establish a chain of custody of the cartridge 10. For example, after drawing a physiological sample as previously discussed herein, a user or the subject may keep the first portion 714 of the adhesive cover 700 in their possession and can send the cartridge 12 with the drawn physiological sample to a laboratory for further analysis. In this embodiment, a computer system 34 that includes or is in communication with a QR code database 36 scans the QR codes 30 and 32.

With reference to FIG. 37, the QR code database 36 includes a plurality of QR codes 38 each of which are associated with a single cartridge 12. In response to scanning the QR codes 30 and 32, the computer system 34 determines if the QR codes are associated with a same cartridge 10 by comparing the scanned QR codes 30 and 32 to the QR codes 38 in the QR code database 36. In response to determining that the QR codes 30 and 32 are associated with the same cartridge 10, the computer system 34 determines that chain of custody has been preserved and outputs a notification indicating chain of custody has been preserved. In response to determining that the QR codes 30 and 32 are associated with different cartridges 10, the computer system 34 determines that chain of custody has not been preserved and outputs a notification indicating chain of custody has not been preserved. In another embodiment, the QR codes 30 and 32 are the same. In this embodiment, the computer system 34 scans the QR codes 30 and 32 and determines that chain of custody has been preserved if the QR codes 30 and 32 are the same. In response to determining that the QR codes 30 and 32 are the same, the computer system 34 outputs a notification that chain of custody has been preserved. In response to determining the QR codes 30 and 32 are different, the computer system 34 outputs a notification that chain of custody has not been preserved.

In another embodiment (FIG. 38), a first computer system 44 is connected to an electronic medical record (“EMR”) database 42 that includes a plurality of EMRs 44 each associated with a different subject. In this embodiment, after drawing a physiological sample from a subject, the first computer system 44 scans the first QR code 30 and sends the cartridge 12 to a laboratory for further analysis. In response to scanning the first QR code 30, the first computer system 40 associates the first QR code 30 and the second QR code 32 with an EMR 44 that corresponds to the subject from which the physiological sample was drawn. In some embodiments, the first computer system 44 may update the associated EMR 44 to indicate that a physiological sample has been drawn automatically or based on a user input. A user of a second computer system 46 receives the cartridge 12 and scans the second QR code 32. The second computer system 46 determines an identity of the subject by matching the QR code 32 to the EMR 44 that is associated with the first and second QR codes 30 and 32. While the above describes a system for identifying a subject from which a physiological sample has been drawn by matching QR codes in an EMR database, the system may identify a subject from which a physiological sample has been drawn by matching QR codes in any database that includes a subject's identity.

As depicted in FIG. 35, the first portion 714 and the third portion 718 of the adhesive cover 700 can also include a serial number 50. The serial number 50 can also be used to establish a chain of custody as previously discussed herein with respect to the QR codes 30 and 32.

Referring now to FIG. 39, a method 800 for obtaining a physiological sample from a subject is disclosed herein.

At 802, a user or a subject removes the first portion 714 of the adhesive cover as previously discussed herein.

At 804 the cartridge 12 of the dermal patch system 10 is affixed to the skin of a subject as previously discussed herein.

At 806, the lancet 100 is engaged with the cartridge 12 to draw a physiological sample (e.g., a blood sample, a sample of interstitial fluid, etc.) from the subject as previously discussed herein.

At 808, the cartridge 12 is removed from the skin of the subject as previously discussed herein.

At 810, the cartridge 12 is sent to a medical professional to analyze the drawn physiological sample as previously discussed herein.

At 812, a computer system determines if chain of custody has been preserved and optionally updates an electronic medical record to indicate a physiological sample has been obtained from a subject as previously discussed herein.

Referring now to FIG. 40, a computer system 900 is shown in accordance with an exemplary embodiment. The computer system 900 may serve as any computer system disclosed herein (e.g., the computer system 40, computer system 46). As used herein a computer system (or device) is any system/device capable of receiving, processing, and/or sending data. Computer systems include, but are not limited to, microprocessor-based systems, personal computers, servers, hand-held computing devices, tablets, smartphones, multiprocessor-based systems, mainframe computer systems, virtual reality (“VR”) headsets and the like.

As shown in FIG. 40, the computer system 900 includes one or more processors or processing units 902, a system memory 904, and a bus 906 that couples the various components of the computer system 900 including the system memory 904 to the processor 902. The system memory 904 includes a computer readable storage medium 908 and volatile memory 910 (e.g., Random Access Memory, cache, etc.). As used herein, a computer readable storage medium includes any media that is capable of storing computer readable; program instructions and is accessible by a processor. The computer readable storage medium 908 includes non-volatile and non-transitory storage media (e.g., flash memory, read only memory (ROM), hard disk drives, etc.). Computer program instructions as described herein include program modules (e.g., routines, programs, objects, components, logic, data structures, etc.) that are executable by a processor. Furthermore, computer readable program instructions, when executed by a processor, can direct a computer system to function in a particular manner such that a computer readable storage medium comprises an article of manufacture. Specifically, the computer readable program instructions when executed by a processor can create a means for carrying out at least a portion of the steps of the methods disclosed herein.

The bus 906 may be one or more of any type of bus structure capable of transmitting data between components of the computer system 900 (e.g., a memory bus, a memory controller, a peripheral bus, an accelerated graphics port, etc.).

The computer system 900 may further include a communication adapter 912 which allows the computer system 900 to communicate with one or more other computer systems/devices via one or more communication protocols (e.g., Wi-Fi, BTLE, etc.) and in some embodiments may allow the computer system 900 to communicate with one or more other computer systems/devices over one or more networks (e.g., a local area network (LAN), a wide area network (WAN), a public network (the Internet), etc.).

In some embodiments, the computer system 900 may be connected to one or more external devices 914 and a display 916. As used herein, an external device includes any device that allows a user to interact with a computer system (e.g., mouse, keyboard, touch screen, etc.). An external device 914 and the display 916 may be in communication with the processor 902 and the system memory 904 via an Input/Output (I/O) interface 918.

The display 916 may display a graphical user interface (GUI) that may include a plurality of selectable icons and/or editable fields. A user may use an external device 914 (e.g., a mouse) to select one or more icons and/or edit one or more editable fields. Selecting an icon and/or editing a field may cause the processor 902 to execute computer readable program instructions stored in the computer readable storage medium 908. In one example, a user may use an external device 914 to interact with the computer system 900 and cause the processor 902 to execute computer readable program instructions relating to at least a portion of the steps of the methods disclosed herein.

Referring now to FIG. 41, a cloud computing environment 1000 is depicted in accordance with an exemplary embodiment. The cloud computing environment 1000 is connected to one or more user computer systems 1002 and provides access to shared computer resources (e.g., storage, memory, applications, virtual machines, etc.) to the user computer systems 1002. As depicted in FIG. 40, the cloud computing environment includes one or more interconnected nodes 1004. Each node 1004 may be a computer system or device local processing and storage capabilities. The nodes 1004 may be grouped and in communication with one another via one or more networks. This allows the cloud computing environment 1000 to offer software services to the one or more computer services to the one or more user computer systems 1002 and as such, a user computer system 1002 does not need to maintain resources locally.

In one embodiment, a node 1004 includes computer readable program instructions for carrying out various steps of various methods disclosed herein. In these embodiments, a user of a user computer system 1002 that is connected to the cloud computing environment may cause a node 1004 to execute the computer readable program instructions to carry out various steps of various methods disclosed herein.

FIG. 43 depicts a bottom layer 300 of the cartridge 12 in accordance with another embodiment of the present disclosure. In this embodiment, the cartridge 12 includes alignment ridges 1102 that can be used to align the bottom layer 300 with the top layer 500. The bottom layer 300 also can include cutout vent holes 1104 that can be used to allow the collected sample on the collection pads 16 to dry faster after collection of the sample. One or more vent holes can be used. These cutout vent holes can also be made in the bottom adhesive layer 200, but not necessarily the liner 14.

FIG. 44 depicts a top layer 500 of the cartridge 12 in accordance with another embodiment of the present disclosure. The top layer 500 includes cutout 1106 for an integrated overflow filter material that can improve the ease of manufacturing.

FIG. 45 depicts a collection pad 16 in accordance with another embodiment of the present. The collection pad 16 can be made as one piece with tabs 1108 formed at a middle point therein, thereby forming a first section 1010 and a second section 1012. The tabs 1108 can be formed as perforations die cut into the pad. The first section 1010 and second section 1012 of the collection pad 16 can be separated by pulling the sections apart and breaking the tabs. The two (2) pieces of the collection pad 16 can be approximately 8 mm×8 mm. Because the collection pad 16 of this embodiment can be made as one piece, as opposed to two (2) pieces that require alignment, it is easier to manufacture the collection pad. However, it is not necessary to manufacture the collection pad 16 as one piece.

The size of the collection pad can be selected to collect a larger physiological sample. For example, the collection pad can be increased from a 70PL capacity (approximately 8 mm×16 mm) to 250 μL capacity (approximately 16 mm×30 mm), as shown in FIG. 46. The size of the pad can be increased to provide a larger collection capacity. This increased volume can be useful in order to separate blood plasma from the blood sample, as presented below. An overflow area can be used in the vicinity of the pad to limit the saturation of blood on the pad in order to keep the collected blood volume within a targeted range of blood volume. As shown in FIG. 46, added material in the periphery of the rectangular pad can be used to draw excess blood from the collection well, thereby preventing over saturation of the rectangular pad, ensuring accuracy of the collected volume as well as assisting with the drying of the sample on the pad for sample viability.

The collection pad can be configured to separate a plasma sample from the physiological/blood sample. A blood sample of approximately 20-250 μL can be used to obtain a plasma sample of approximately 5-200 μL. This can be done by including a plasma filter in the collection pad that includes a separator that separates the plasma from the blood. For example, a Whatman Fusion 5 pad can be used in place of the collection pad 16. As described above with reference to collection pad 16 in FIGS. 13 and 25, viewing apertures can be aligned with the Fusion 5 pad, which can be covered with a transparent film that allows a user of the dermal patch system to view the Fusion pad in order to visually confirm blood plasma collection. Also, the cover can include a transparent portion that allows a user to view the plasma collection pad. Similar to the collection pad 16 described above, the Fusion 5 pad can be sent to a lab for analysis of the plasma. Upon receipt of the sample, a section containing the separated plasma can be torn away.

An example plasma filter 1200 is shown in FIG. 47. The filter 1200 includes a blood separation portion 1210 and a conjugate release portion 1220. In the blood separation portion 1210, a vertical flow separator or a lateral flow separator can be used to separate the plasma from the blood. For example, an LF1 separator can be used for lateral flow assays, and works well with one drop of blood (approximately 10-15 μL). Also, an MF1 separator can be used for lateral or vertical flow assays for a blood volume of about 100 μL, or 15-50 μL. A VF2 vertical separator can be used as a single or multiple layers for separation of a wide range of blood volumes (e.g., >50 μL). A Fusion 5 separator can be used as a lateral flow blood separator using two drops of blood (approximately 15-50 μL), and can be used for conjugate release. A Whatman GF/DVA bound glass separator can be used for separation of a wide range of blood volumes (e.g., >50 μL). A Fusion 5 separator also can be used for conjugate release in the conjugate release portion 1220. Alternatively, an Std 17 or Std 14 can be used for the conjugate release. A Std 17 conjugate release has a higher absorbency than a Std 14 conjugate release. Also, GF, GX or GR Vivid Plasma Separation Membranes, a Cytosep Membrane Media Grade 1660 or an MDI Membrane Technologies FR1 (IPD) can be used for plasma separation.

FIG. 48 shows a sample collection pad that can be used to collect plasma. As shown in FIG. 48, the plasma collection pad can include several layers including top and bottom clear laminate layers that can be used to seal the top and bottom extraction zones, respectively. Below the top clear laminate layer is a polyester membrane that can be used to remove white blood cells (WBC's). Below the polyester membrane is a first double-sided adhesive that adheres to the polyester membrane. Below the first double-sided adhesive is an asymmetric polysulfone layer that is adhered thereto and can be used to remove red blood cells (RBC's). Below the asymmetric polysulfone layer is a second double-sided adhesive that adheres to the asymmetric polysulfone layer. Below the second double-sided adhesive is a wax-patterned cellulose (TFN) layer that provides a wicking source and plasma storage. Below the wax-patterned cellulose (TFN) layer is a third double-sided adhesive that adheres the wax-patterned cellulose (TFN) layer to the bottom clear laminate layer. When a blood sample is applied to the plasma collection, plasma will be separated from the blood sample and stored in the wax-patterned cellulose (TFN) layer. The plasma collection pad can be covered with a transparent film that allows a user of the dermal patch system to view the collection pad in order to visually confirm blood plasma collection through viewing apertures. Also, the cover can include a transparent portion that allows a user to view the plasma collection pad.

As previously discussed, the above may be implemented by way of computer readable instructions, encoded or embedded on computer readable storage medium (which excludes transitory medium), which, when executed by a processor(s), cause the processor(s) to carry out the methods of the present disclosure.

While various embodiments have been illustrated and described in detail in the drawings and foregoing description, such illustration and description are to be considered illustrative or exemplary and not restrictive; embodiments of the present disclosure are not limited to the disclosed embodiments. Other variations to the disclosed embodiments can be understood and effected by those skilled in the art in practicing embodiments of the present disclosure, from a study of the drawings, the disclosure, and the appended claims.

In the claims, the word “comprising” does not exclude other elements or steps, and the indefinite article “a” or “an” does not exclude a plurality. A single processor or other processing unit may fulfill the functions of several items recited in the claims. The mere fact that certain measures are recited in mutually different dependent claims does not indicate that a combination of these measured cannot be used to advantage. Any reference signs in the claims should not be construed as limiting the scope.

What is claimed is:

1. A system for collecting a physiological sample from a subject comprising:
a lancet configured to draw a physiological sample, and
a cartridge configured to be affixed to the subject's skin and including:
a physiological sample well,
a sloped physiological sample channel in open communication with the physiological sample well,
a sample collection pad in open communication with the sloped physiological channel and configured to absorb the drawn physiological sample,
wherein the physiological sample well is configured to retain the drawn physiological sample and the sloped physiological channel is configured to carry the drawn physiological sample from the physiological sample to the sample collection pad.

2. The system of claim 1, further comprising:
a desiccant disposed within the cartridge.

3. The system of claim 1, further comprising:
a hydrophobic foam disposed within the cartridge and configured to prevent the drawn physiological sample from escaping the sample collection pad.

4. The system of claim 1, wherein the sample collection pad is a first sample collection pad and the cartridge includes a second collection pad, and
wherein the physiological sample channel is configured to carry the drawn physiological sample to the first and second collection pad.

5. The system of claim 1, wherein the lancet includes:
one or more needles configured to move between an undeployed position and a deployed position, wherein the one or more needles are disposed within the lancet when in the undeployed position and extends out of the lancet when in the deployed position to draw the physiological sample.

6. The system of claim 5, wherein the cartridge includes:
a lancet receiving element,
wherein the lancet is configured to move the one or more needles from the undeployed position to the deployed position when the lancet engages with the lancet receiving element.

7. The system of claim 5, wherein the lancet is configured to automatically retract the one or more needles into a housing of the lancet.

8. The system of claim 1, further comprising:
a vacuum pin disposed within the cartridge and configured to move between a deployed position and an undeployed position,
wherein the vacuum pin is configured to create a vacuum within the cartridge when moved from the undeployed position to the deployed position and the vacuum draws the drawn physiological sample from the physiological sample well to the sample collection pad.

9. The system of claim 1, wherein the cartridge includes:
a first layer of material, and
a second layer of material,
wherein the first and second layer of material define the physiological sample channel.

10. A system for collecting a physiological sample from a subject comprising:
a lancet configured to draw a physiological sample,
a cartridge configured to receive and store the physiological sample including:
a cover with a first portion and a second portion, wherein the first portion is removable,
a first quick response code disposed on the first portion of the cover,
a second quick response code disposed on the second portion,
wherein the first and second quick response codes are associated with the cartridge.

11. The system of claim 10, wherein the cartridge further includes:
a sample collection pad,
wherein the cover seals the sample collection pad within the cartridge.

12. The system of claim 11, wherein the cover includes a transparent portion that allows a user to view the sample collection pad.

13. The system of claim 12, wherein the cover includes a third portion that covers the sample collection pad and includes the transparent portion, wherein the third portion is removable.

14. The system of claim 10, wherein the lancet includes one or more needles that are moveable between an undeployed position and a deployed position, wherein the one or more needles is configured to draw the physiological sample when in the deployed position, and
the cartridge further includes:
a lancet receiving element configured to cause the lancet to move the one or more needles to the deployed position,
wherein the first portion covers the lancet receiving element.

15. The system of claim 10, wherein the first portion includes a pull tab which allows a user to remove the first portion.

16. The system of claim 10, wherein the cartridge includes:
a moveable vacuum pin configured to create a vacuum within the cartridge when moved, and
wherein the vacuum draws the physiological sample to the sample collection pad.

17. A method for drawing a physiological sample comprising:
affixing a cartridge of a dermal patch system to the skin of a subject,
engaging a lancet with the cartridge to draw a physiological sample,
removing the cartridge from the skin of the subject, and
determining if chain of custody of the cartridge has been preserved by scanning a quick response code of the cartridge.

18. The method of claim 17, wherein the lancet includes one or more needles that are configured to move from an undeployed position to a deployed position upon engagement with the cartridge.

19. The method of claim 17, wherein lancet is configured to automatically retract the one or more needles.

20. The method of claim 17, further comprising:
pulling a vacuum pin of the cartridge to draw the physiological sample to a sample collection pad of the cartridge.

21. The system of claim 1, wherein sample collection pad comprises sections with tabs therebetween.

22. The system of claim 10, wherein sample collection pad comprises sections with tabs therebetween.

23. The method of claim 17, wherein sample collection pad comprises sections with tabs therebetween.

24. The system of claim 1, wherein the cartridge comprises a bottom layer, an adhesive layer attached to the bottom layer, a top layer, a sample well opening formed in the adhesive layer, a needle aperture formed in the top layer, wherein the needle aperture is smaller than the sample well opening.

25. The system of claim 6, wherein the lancet is configured to move the one or more needles from the undeployed position to the deployed position only when a configuration of the lancet engages with a matching configuration of the cartridge.

26. The system of claim 6, wherein the lancet is configured to move the one or more needles from the undeployed position to the deployed position automatically when a configuration of the lancet engages with a matching configuration of the cartridge.

27. The system of claim 24, further comprising a desiccant formed in a space between the top layer and the bottom layer.

28. The system of claim 24, wherein the adhesive layer is formed of a pressure sensitive adhesive.

29. The system of claim 5, wherein the one or more needles are coated with silicone.

30. The system of claim 14, wherein the one or more needles are coated with silicone.

31. The system of claim 24, wherein the cartridge includes alignment ridges configured to align the bottom layer with the top layer.

32. The system of claim 24, wherein the bottom layer includes at least one cutout vent hole configured to allow the physiological sample on the sample collection pad to dry faster.

33. The system of claim 24, wherein the adhesive layer includes at least one cutout vent hole configured to allow the physiological sample on the sample collection pad to dry faster.

34. The system of claim 24, wherein the top layer includes a cutout configured to hold an integrated overflow filter.

35. The system of claim 1, wherein the sample collection pad is made from one piece having tabs formed at a middle portion thereof, thereby forming a first section and a second section that are configured to be separated from each other when the first and second section are pulled apart.

36. The system of claim 35, wherein the first section and the second section each are approximately 8 mm by 8 mm.

37. The system of claim 24, wherein the first section and the second section each are approximately 16 mm by 15 mm.

38. The system of claim 1, wherein the sample collection pad is configured to separate a plasma sample from the physiological sample.

39. The system of claim 38, wherein the sample collection pad comprises a blood separation section and a conjugate release section.

40. The system of claim 39, wherein the blood separation section comprises a separator selected from a group consisting of an LF1 separator, an MF1 separator, a VF2 vertical separator, a Fusion 5 separator, and GF/DVA bound glass separator.

41. The system of claim 39, wherein the conjugate release section comprises a conjugate release element selected from a group consisting of a Std 14, Std 17 and Fusion 5.

42. The system of claim 38, wherein the sample collection pad comprises a plasma separation stack up.

43. The system of claim 42, wherein the plasma separation stack up comprises a membrane configured to remove white blood cells, a membrane configured to remove red blood cells and a layer configured to provide a wicking source and plasma storage.

44. The system of claim 43, wherein the plasma separation stack up comprises top clear laminate layer configured to allow viewing of the plasma sample, a polyester membrane located below the top clear laminate layer and configured to remove white blood cells, a first double-sided adhesive located below the polyester membrane and configured to adhere to the polyester membrane, an asymmetric polysulfone layer located below the first double-sided adhesive and configured to remove red blood cells, a second double-sided adhesive located below the asymmetric polysulfone layer and configured to adhere to the asymmetric polysulfone layer, a wax-patterned cellulose layer located below the second double-sided adhesive and configured to provide a wicking source and plasma storage, a third double-sided adhesive located below the wax-patterned cellulose layer and configured to adhere to the wax-patterned cellulose layer, and a clear laminate bottom layer located below the third double-sided adhesive.

45. The system of claim 1, further comprising an overflow area in the vicinity of the pad configured to limit the saturation of blood on the pad in order to keep the collected blood volume within a targeted range.
